(19) United States
(12) Patent Application Publication
(10) Pub. No.: US 2003/0175968 A1
(43) Pub. Date: Sep. 18, 2003

(54) Gene targeting method

(76) Inventors: Kent G. Golic, Salt Lake City, UT (US); Yikang S. Rong, Salt Lake City, UT (US); Gary N. Drews, Salt Lake City, UT (US)

Correspondence Address: Greenlee Winner & Sullivan, Suite 201, 5370 Manhattan Circle, Boulder, CO, 80303, US

(21) Appl. No.: 10/204,039

(22) PCT Filed: Mar. 1, 2001
(86) PCT No.: PCT/US01/07051

(51) Int. Cl. C12N 15/85; C12N 15/82

(52) U.S. Cl. 435/455; 435/468

(57) ABSTRACT

The invention related to a method of gene targeting in a transformable host organism, and compositions useful for carrying out the method. The method of gene targeting provides improvement over previous gene targeting methods since it is generally applicable over a wide variety of transformable organisms. It provides time savings in producing organisms with specific gene modifications, and it does not require a pluripotential cell line. The targeting method of the invention exploits the endogenous cellular process of homologous recombination to implement gene targeting at essentially any known gene.

### CROSS REFERENCE TO RELATED APPLICATIONS

This application claims priority from U.S. Provisional Patent Application Nos. 60/258,682 filed Dec. 28, 2000, 60/188,672, filed Mar. 13, 2000, and 60/187,220, filed Mar. 3, 2000.

### ACKNOWLEDGMENT OF FEDERAL RESEARCH SUPPORT

The U.S. Government has certain rights in the invention based upon partial support by grant R21GM57792 from the National Institutes of Health, U.S. Public Health Service.

### BACKGROUND OF THE INVENTION

When exogenous DNA or RNA is introduced into a cell, the cell is said to be transformed. Various methods are known by which the transforming nucleic acid becomes a permanent part of the transformed cell's genome. Unless specialized methods are used, permanent transformation is usually the result of integration of the transforming nucleic acid in chromosomal DNA at a random location. The transforming DNA can also be introduced into the cell on a plasmid that replicates autonomously within the cell and which segregates copies to daughter cells when the cell divides. Either way, the locus of the transforming nucleic acid with respect to endogenous genes of the cell is unspecified. Gene targeting is the general name for a process whereby chromosomal integration of the transforming DNA at a desired genetic locus is facilitated, to the extent that permanently transformed cells having the DNA at that locus can be obtained at a useful frequency. Typically, the gene at the target locus is modified, replaced or duplicated by the transforming (donor) nucleic acid. integration events that have occurred (or select against undesired integration events). Without such steps, the desired integration might occur by chance, but with such a low frequency as to be undetectable.

Yeast (Saccharomyces cerevisiae) has been a useful organism for development of gene targeting methods. Rothenstein, R. (1991) Methods in Enzymology 194:281-301 reviewed techniques of targeted integration in yeast. The normal yeast process of homologous recombination was shown to permit integration of transforming plasmid DNA having a segment of sequence homologous to a yeast gene. When a double-strand break was introduced within a homologous segment, transformation with the resulting linear DNA resulted in a 10-1000-fold increased incidence of integration at or near the break The longer the region of homology on either side of the break, the greater the frequency of recombination at the desired locus. Strategies for gene replacement, gene disruption and rescue of mutant alleles were described.

The studies of gene targeting in yeast have been facilitated by the fact that individual transformed cells can be isolated and grown in pure culture to any convenient amount. In addition, the short doubling time of yeast cells in culture has allowed researchers to observe events that occur with a low frequency and to study the genetics of those events within a convenient time scale. When working with complex multicellular organisms, the number of individuals which can be assessed for a genetic change, and the time scale required for observing patterns of inheritance are both increased. To achieve practical gene targeting in such organisms, techniques were developed to increase the frequency of observable targeting events and to increase the efficiency of selection for desired events. Practical methods of gene targeting have been developed in the fruit fly, Drosophila melanogaster, and in the mouse, Mus musculus, however such methods have not been applicable to a wider range of organisms.

Transposons have been utilized for inducing gene targeting in Drosophila. Gloor, G. B., et al. (1991) Science, 253:1110-1117 described utilizing the property of the P element transposon to generate a double strand gap when a transposition event occurs, the gap being located at the site formerly occupied by the transposon. Under most circumstances the resulting gap is repaired by copying from homologous sequences on the sister chromatid. If a homologous sequence is present in the cell at an ectopic locus, for example on a plasmid, that sequence can also serve as a template to repair the double strand gap generated by the transposon's departure. This type of gap repair can then be employed to target a desired sequence to the locus of the departing transposon. The primary limitation of the process is that the host organism must have a transposon located at or near the target site.

The FLP-FRT recombinase system of yeast was employed to mobilize FRT-flanked donor DNA and generate re-integration at a different chromosomal location (Golic, M. M., et al, (1997) Nucl. Acids Res. 25:3665-3671). The donor DNA was introduced into the Drosophila chromosome flanked by repeats of the FRT recombinase recognition site, all within a P element for integration. The FLP recombinase was introduced under control of a heat-shock promoter, so that the enzyme could be activated by the investigators at a specified time. The action of FLP recombinase could result in excision of the donor DNA followed by a second round of recombination at a target site where another FRT site was present. The phenomenon could be observed by using flies having the target FRT site at the locus of a known gene where an altered phenotype was detectable.

Gene targeting in mammals has only been achieved to any significant degree in the mouse. Uniquely in the case of the mouse, a pluripotent cell line exists, embryonic stem (ES) cells that can be grown in culture, transformed, selected and introduced into an embryonic stage, the blastocyst stage of the mouse embryo. Embryos bearing inserted transgenic ES cells develop as genetically chimeric offspring. By interbreeding siblings, homozygous mice carrying the selected genes can be obtained. An overview of the process and its limitations is provided by Capecchi, M. R, (1989) Trends in Genetics 5:70-76; and by Bronson, S. K. (1994) J. Biol. Chem. 269: 27155-25158. Both homologous and non-homologous recombination occur in mammalian cells. Both processes occur with low frequency and non-homologous recombination occurs more frequently than homologous recombination. ES cells are transfected with a DNA construct that combines a donor DNA having the modification to be introduced at the target site combined with flanking sequence homologous to the target site, and marker genes, as needed, for selection, as well as any other sequences that may be desired. The donor construct need not be integrated into the chromosome initially, but can recombine with the target site by homologous recombination or at a non-target site by non-homologous recombination. Since these events are rare, dual selection is required to select for recombinants and to select against non-homologous recombinants. The selections are carried out in vitro on the ES cells in culture. PCR screening can also be employed to identify desired recombinants. The frequency of homologous recombination is increased as the length of the region of homology in the donor is increased, with at least 5 kb of homology being preferred. However homologous recombination has been observed with as little as 25-50 bp of homology. Donor DNA having small deletions or insertions of the target sequence are introduced into the target with higher frequency than point mutations. Both insertions of sequence and replacement of the target, as well as duplication in whole or in part of the target can be accomplished, by appropriate design of the donor vector and the selection system, as desired for the purpose of the targeting.

Gene targeting in mammals other than the mouse has been limited by lack of ES cells capable of being transplanted and of contributing to germ line cells of developing embryos. However techniques related to cloning technology have opened new possibilities for extending targeting to other species. McCreath, K. J., et al (2000) Nature 405:1066-1069 have reported successful targeting in sheep by carrying out transformation and targeting selection in primary embryo fibroblast cells. The targeted fibroblast nuclei were then transferred to enucleated egg cells followed by implantation in the uterus of a host mother. The technique provides the advantage that the generation of chimeric animals and subsequent breeding to homozygosity are not required. However the time available for carrying out targeting and selection is short.

The use of recombinases and their recognition sites has proven to be a valuable tool once the initial targeting event has been achieved. For a review of the techniques applying the site specific recombinase systems, see Sauer, B. et al, (1994) Current Opinion in Biotech. 5:521-527. See also U.S. Pat. No. 4,959,317. For example, repeated targeting at a given locus is facilitated by including recombination-specific recombination sites in the initial targeting construct. Once in place, the recombination sites can be used, in combination with their respective recombinase, to provide highly efficient transfer of an exogenous DNA to the locus of the recombination site. A recombinase system commonly used is the Cre recombinase, which recognizes a sequence designated loxP. The Cre recombinase and loxP recognition site are derived from bacteriophage P1. Another widely used system, derived from the 2μ circle of Saccharomyces cerevisiae, is the FLP recombinase which recognizes a specific sequence, FRT. In both systems, the effect of recombinase activity is determined by the orientation of the recognition sites flanking a given segment of DNA. A DNA sequence flanked by directly repeated recombination sites and then integrated into the genome by either homologous or illegitimate recombination can subsequently be removed simply by providing the corresponding recombinase. One useful consequence of this property has been exploited to remove an unwanted selection marker from the target site once homologous recombination has occurred and selection is no longer necessary. In another application, a gene which may exert a toxic effect can be maintained in a dormant state by inserting a lox-flanked sequence between the promoter and the gene, the sequence being designed to prevent expression of the gene. Expression of Cre activity results in excision of the intervening sequence and allows to promoter to act to activate the dormant gene. Cre can be introduced by mating or provided in an inducible form that permits activation at the investigator's control. A variety of other post-targeting strategies can be facilitated by the use of site specific recombination systems, as known in the art.

As has been shown in yeast, introducing a ds break into DNA increases recombination frequency. A number of studies have demonstrated that introducing a ds break into a target site increased recombination with a homologous donor DNA about 100-fold. The ds break was created by providing an I-SceI site in the target DNA, then introducing and expressing an I-SceI endonuclease along with a donor DNA homologous to the target. Using Chinese hamster ovary (CHO) cells, Sargent, R. G. et al (1997) Mol. Cell. Biol. 17:267-277 described an experiment for testing crossovers between tandem repeats of an APRT gene, one of which carried an I-SceI site. The occurrence of homologous recombination could be measured by crossovers between the tandem APRT loci, which eliminated an intervening thymidine kinase (Tk⁺) gene, or within different segments of the APRT gene itself, based on the presence or absence in the progeny, of certain mutations located in one of the tandem genes. A ds break was generated at the I-SceI site by introducing and expressing the I-SceI endonuclease carried on a separate expression vector and introduced by transformation. A similar type of demonstration was reported by Liang, F. et al (1998) Proc. Natl. Acad. Sci. USA 95:5172-5177. Cohen-Tannoudji, M. et al (1998) Mol. Cell. Biol. 18:1444-1448 described the use of an I-SceI site introduced into a target gene by conventional targeting. Once in place, other constructs could be introduced at the same target (“knocked in”) by a subsequent transformation with a desired donor construct and transient expression of I-SceI endonuclease to introduce a ds-break at the target. The efficiency of the second targeting step was reportedly 100-fold greater than was observed for conventional targeting. The method had the disadvantage that an I-SceI site was required at the target site.

U.S. Pat. No. 5,962,327 describes the I-SceI endonuclease and its recognition site. The patent also discloses general strategies using I-SceI that can be attempted for the site-specific insertion of a DNA fragment from a plasmid into a chromosome. A diagram of site-directed homologous recombination in yeast is presented. It should be noted that this technique was shown only in yeast.

In plants, spontaneous homologous recombination events have been characterized as “extremely rare” (Puchta, H. (1999) Genetics 199:1173-1181). Introduction of ds-breaks has been shown to increase the homologous recombination frequency. Puchta, H. et al (1996) Proc. Natl. Acad. Sci. USA 93:5055-5060 reported introducing (by T-DNA mediated transformation) a target locus bearing an I-SceI site and a partial kanamycin resistance gene. In a second round of transformation, a repair construct was introduced along with an I-SceI expression cassette. Homologous recombination to restore kanamycin resistance was detected by the presence of kanamycin-resistant callus cells.

### SUMMARY OF THE INVENTION

The present invention includes methods and compositions for carrying out gene targeting. Unlike previously known methods for gene targeting in multicellular organisms, the present invention does not depend on availability of a pluripotential cell line, and hence can be adapted for gene targeting in any organism. The method exploits homologous recombination processes that are endogenous in the cells of all organisms. Any gene of an organism can be modified by the method of the invention as long as the sequence of the gene, or a portion of the gene, is known, or if a DNA clone is available.

“Target” is the term used herein to identify the genetic element or DNA segment to be modified. “Donor” is used herein to identify those genetic elements or DNA segments used to modify the target. The modification can be any sort of genetic change, including substitution of one segment for another, insertion of single or multiple nucleotide replacements, deletion, insertion, duplication of all or part of the target, and combinations thereof.

In general outline, a donor construct is provided within cells of the organism. The donor construct can be integrated anywhere in the genome, without regard to the locus of the target. Alternatively, the donor construct can be carried on an autonomously replicating genetic element, or present transiently. The donor construct includes a version of the target, the target modifying sequence, containing any sequence modifications to be introduced at the target site and also having a unique endonuclease site. Action of an endonuclease able to recognize the unique site results in a double strand break within the modifying sequence, generating a recombinogenic donor. Prior to, or in combination with, generating the double strand break, the donor construct is excised from its locus of integration, by various means described hereinafter. The combination of the excision and endonuclease cutting frees the recombinogenic donor to undergo homologous recombination at the target site resulting in the desired genetic change at the target. If the donor construct is not chromosomally integrated, but merely present on a plasmid in the host cell, the excision step is not needed. As described herein, the use of various selectable markers at specified positions of the donor construct relative to the modifying sequence facilitates identifying recombinants and selecting for the desired type of recombinant.

The timing of the excision and endonuclease steps is controlled by maintaining the enzymes that catalyze these reactions under inducible or tissue-specific expression control. The genes encoding the enzymes combined with their promoters or MRNA encoding the enzymes or the enzymes themselves can be introduced to the organism concomitantly with the donor construct. Alternatively, a transgenic strain of the organism carrying the genes can be provided by a prior step of transformation and selection. Such a strain is termed herein a carrier host organism. A carrier host organism is useful as a host for all desired target gene modifications of the host species.

Many alterations and variations of the invention exist as described herein. The invention is exemplified for gene targeting in the insect, Drosophila, and in the plant, Arabidopsis. In both these organisms nucleotide sequences are known for most of the genome. Increasingly larger segments of genomic sequences are becoming known for a growing number of organisms. The functional elements used to carry out the steps of the invention are known for any desired organism. Therefore the present invention can be adapted for application in any organism. The invention therefore provides a general method for gene targeting in any organism, as well as a method for making a carrier host strain of any organism. Products of the invention include transformation vectors for gene targeting that include a modifying sequence having a unique endonuclease recognition site associated therewith such that endonuclease cutting at the site yields a recombinogenic donor. The invention also provides a transformation vector for generating a carrier host organism including an endonuclease capable of making double strand break in DNA at the unique site, the endonuclease being under control of an inducible promoter.

### DESCRIPTION OF THE DRAWINGS

FIG. 1 is a diagram demonstrating I-SceI cutting efficiency (Example 1). The reporter constructs were transformed via P elements (indicated by small arrowheads), and carried the I-SceI cut site (as indicated) either (A) adjacent to a shortened version of the wild type w⁺ gene (indicated by the large solid arrow), or (B) flanked by a complete copy and a non-functional partial copy of that w⁺ gene. The complete gene is ˜4.5 kb in length and the non-functional partial gene is ˜3.5 kb.

FIG. 2 is a diagram showing the construct for yellow targeting. At the top is diagramed the donor construct (P[y-donor]) as it would appear in the chromosome when initially transformed via P element transformation. Diagramed beneath that is the form of the extrachromosomal donor DNA after FLP-mediated excision and I-SceI cutting. The arrow indicates transcriptional direction of yellow. Cut site: 18 bp I-SceI recognition sequence, β2t:β2t tubulin gene.β3t: coding region of β3 tubulin gene. S: restriction site for SalI. Underlines indicate the DNAs used as probes for chromosome in situ hybridization and Southern blot analyses.

FIG. 3 is a diagram of gene targeting configurations. Two typical forms of gene targeting constructs are shown, and the results of their recombination with the target locus.

FIG. 4 is a diagram of crossing schemes for yellow rescue (Example 2).

FIG. 5 shows cytological localization of a targeted insertion. The cytological positions of β2t hybridization are indicated on the chromosomes of this y¹/y⁺ Class III female.

FIG. 6 is a diagram showing types of targeting events. The four classes of recovered targeting events are shown, with the likely mechanism of origin for each indicated at the left, and the product of each event at the right. The donor construct is diagramed as in FIG. 2. The approximate position of the point mutation in y¹ is indicated by an asterisk. The expected sizes of the DNA fragments produced by SalI digestion are shown below each product at the right. the presumed allelomorphs of y are indicated above each copy of the gene. The approximate locations of the insertions [symbol] and deletions (Δ) found in Class III events are indicated.

FIG. 7 provides results of Southern blot analyses of targeting events. Roman numerals indicate the type of targeting event by class type. Lanes 1 and 13 are controls: C1 is DNA from y¹ males; C2 is DNA from y¹ males that also carry the donor construct shown in FIG. 2.

FIG. 8 is a diagram of gene knock-out by targeting with a truncated gene. The donor DNA used for targeting consists of a truncated gene, missing portions at both the 5′ and the 3′ ends. Donor integration disrupts the endogenous gene by splitting it into two pieces, each having a deletion of a different part of the gene.

FIG. 9 is a diagram of a two-step method for introducing a mutation into a target zone. I-CreI is a rare-cutting endonuclease.

FIG. 10 is a diagram of a donor construct for gene targeting in plants transformed via T-DNA. “kanR” denotes a kanamycin resistance marker gene. “GFP” is a green fluorescent protein marker gene.

FIG. 11 is a diagram of a donor construct designed for targeting using a transposase to excise the recombinogenic donor.

FIG. 12 is a diagram of a donor construct designed for carrying out the steps of the invention using a recombinase and a transposase.

FIG. 13 is a diagram of a donor construct designed for carrying out the invention using a transposase and a site-specific endonuclease.

FIG. 14 shows pug targeting mechanism. The extrachromosomal targeting molecule produced by FLP excision and I-SceI cutting is shown at the top. The endogenous pug⁺ locus is shown in the middle with the direction of transcription being from left to right. The genomic structure resulting from homologous recombination is depicted at the bottom. The probe used in Southern blot analysis (FIG. 15) and selected restriction fragments are shown with sizes indicated in kb. Restriction sites are R: EcoRI, B: BamHI.

FIG. 15 shows Southern blot analysis of a pug targeting event. Fly DNA was digested with EcoRI and BamHI. The membrane was hybridized with a 2.5 kb pug probe (FIG. 14). Lane 1: molecular markers with indicated sizes. Lane 2: pug⁺ control showing the endogenous 9 kb band. Lane 3: DNA from flies homozygous for the targeted pug allele showing, as predicted, the 7 kb and the 10 kb fragments.

FIG. 16 is a diagram showing steps for generating a null mutation of a Target Gene TG). The top line shows both the donor construct, shown as a loop having a lox gene, an I-CreI site (C), a first flanking homologous segment (FH-1) shown with a gap to indicate an I-SceI site, and a second flanking homologous region (FH-2) aligned with a segment of the genome, shown as a straight line having TG flanked by FH-1 and FH-2. The second line diagrams the structure after I-SceI cutting and homologous recombination in the FH-1 region. The third line diagrams an alignment of segments of the structure of line two after I-CreI cutting. The bottom line diagrams the resulting genomic structure after homologous recombination within FH-2.

FIG. 17 is a diagram of a donor construct (top line) structured for ends-in targeting using a combination of transposase and unique endonuclease. Transposase-recognizable inverted repeats (IR), I-SceI site (1), target gene modifying sequences (TGMS) and selectable marker gene (SMG) are identified. The bottom line shows the alignment of the recombinogenic donor and the target after transposase and endonuclease action.

FIG. 18 is a diagram of targeting using a donor construct (top line) having two I-SceI sites (1) but no recombinase or transposase recognition sites. Other abbreviations as in FIG. 17. DR direct repeat.

FIG. 19 is a diagram of targeting by the ends-out method through y¹ rescue.

FIG. 20 is a diagram of ends-out replacement.

FIG. 21 is a diagram of the targeting vector pTV2.

FIG. 22 is a diagram showing a simplified targeting screen.

FIG. 23 is a diagram of a crossing scheme used to eliminate the mapping and marking steps as a prerequisite for targeting.

FIG. 24 is a diagram showing that the stable transformant step can be bypassed and somatic cell nuclei can be used to generate clones: yellow+clones in somatic cells of flies after coinjection of yellow donor DNA and I-SceI encoding MRNA.

### DETAILED DESCRIPTION OF THE INVENTION

The present invention relates to methods and compositions for carrying out gene targeting. In contrast previously known methods for gene targeting in multicellular organisms, the present invention does not depend on availability of a pluripotential cell line, and is adaptable to any organism. Any gene of an organism can be modified by the method as the method exploits homologous recombination processes that are endogenous in the cells of all organisms.

The methods of gene targeting of the invention fall into two general categories which both rely on homologous recombination: (A) the release only method, and (B) the release and cut method. Both methods involve the transformation of an organism with a donor construct of the invention. The release only method can be implemented through a variety of embodiments, including but not limited to, flanking a target gene and optional marker gene(s) in the donor construct with (1) transposons, (2) rare-cutting endonuclease sites, and (3) a transposon and rare-cutting endonuclease site. The release and cut method can be implemented through a variety of embodiments, including but not limited to, flanking a target gene and optional marker gene(s) in the donor construct with (1) site-specific recombinase target sites and cutting with a rare-cutting endonuclease, and (2) site-specific recombinase target sites and cutting with transposons. Other schemes based on these general concepts are within the scope and spirit of the invention, and are readily apparent to those skilled in the art.

The following terms are used herein according to the following definitions.

“Gene targeting” is a general term for a process wherein homologous recombination occurs between DNA sequences residing in the chromosome of a host cell or host organism and a newly introduced DNA sequence.

“Host organism” is the term used for the organism in which gene targeting according to the invention is carried out.

“Target” refers to the gene or DNA segment subject to modification by the gene targeting method of the present invention. Normally, the target is an endogenous gene, coding segment, control region, intron, exon, or portion thereof, of the host organism. The target can be any part or parts of genomic DNA.

“Target gene modifying sequence” is a DNA segment having sequence homology to the target but differing from the target in certain ways, in particular with respect to the specific desired modification(s) to be introduced in the target.

“Unique endonuclease site” is a recognition site for an endonuclease that catalyzes a double strand break in DNA at the site. Any recognition site that does not otherwise exist in the host organism, or does not exist at a site where double-strand breakage is harmful to the host organism, can serve as a unique endonuclease site for that organism. “Unique” is therefore an operational term. Furthermore, modified host organisms may be generated in which an endogenous site or sites have been modified so that they are no longer recognized by the endonuclease. Such a modified host organism can be generated by expressing the endonuclease in the organism and selecting for individuals that are resistant to harmful effects of such expression. Such resistant individuals can arise by cutting followed by inaccurate repair of the break and consequent alteration of the recognition sequence. Alternatively, within a population of individuals, pre-existing polymorphisms may already exist and be selected for by expression of the endonuclease. Many classes of enzymes catalyze double-strand DNA breakage in a site-specific manner, identified by a specific nucleotide sequence at or near the break point. Such enzymes include, but are not limited to transposases, recombinases and homing endonucleases. By introducing the nucleotide sequence of a unique endonuclease site into a donor construct, a double-strand break can be generated at or near that site by action of the appropriate endonuclease. A preferred class of unique endonuclease sites of practical utility are the homing endonuclease or rare-cutting endonuclease sites. The rare-cutting endonuclease sites are typically much longer than restriction endonuclease sites, usually ten or more base pairs in length and thus occur rarely, if at all, in a given host organism. For a review of the rare-cutting endonucleases and details of their recognition site sequences see Belfort, M., et al, (1997) Nucl. Acids Res. 25:3379-3388, incorporated herein by reference. Some of the rare-cutting endonucleases are encoded by organelle genomes, and the coding sequences may use non-standard coding. The coding sequences of many such endonucleases are known and have, or can be, modified to be expressible from a chromosomal locus. The expression can be controlled, if desired, by an inducible promoter. In principle, any rare-cutting endonuclease can be employed in the practice of the invention, including, for example I-CreI, I-SceI, I-Tli, I-CeuI, I-PpoI and PI-PspI.

“Marker” is the term used herein to denote a gene or sequence whose presence or absence conveys a detectable phenotype of the organism. Various types of markers include, but are not limited to, selection makers, screening markers and molecular markers. Selection markers are usually genes that can be expressed to convey a phenotype that makes the organism resistant or susceptible to a specific set of conditions. Screening markers convey a phenotype that is a readily observable and distinguishable trait. Molecular markers are sequence features that can be uniquely identified by oligonucleotide probing, for example RFLP (restriction fragment length polymorphism), SSR markers (simple sequence repeat), and the like.

“Donor construct” is the term used herein to refer to the entire set of DNA segments to be introduced into the host organism as a functional group, including at least the modifying sequence(s), one or more unique endonuclease sites, one or more markers, and optionally one or more recombinase target sites as well as other DNA segments as desired. In one embodiment of the invention, the donor construct is flanked by transposon target sites so that the donor construct becomes integrated somewhere in the host genome after being introduced into host cells. An excisable donor construct is one which can be excised (freed) from its location on the host chromosome or on an extrachromosomal plasmid, by the action of an inducible enzyme, for example, a unique restriction enzyme or a recombinase. In order to be excisable, the donor construct must be flanked by recognition sites for the excising enzyme. For example, in the upper diagram of FIG. 2, the donor construct is flanked by FRT sites which render the construct excisable by the Flp recombinase.

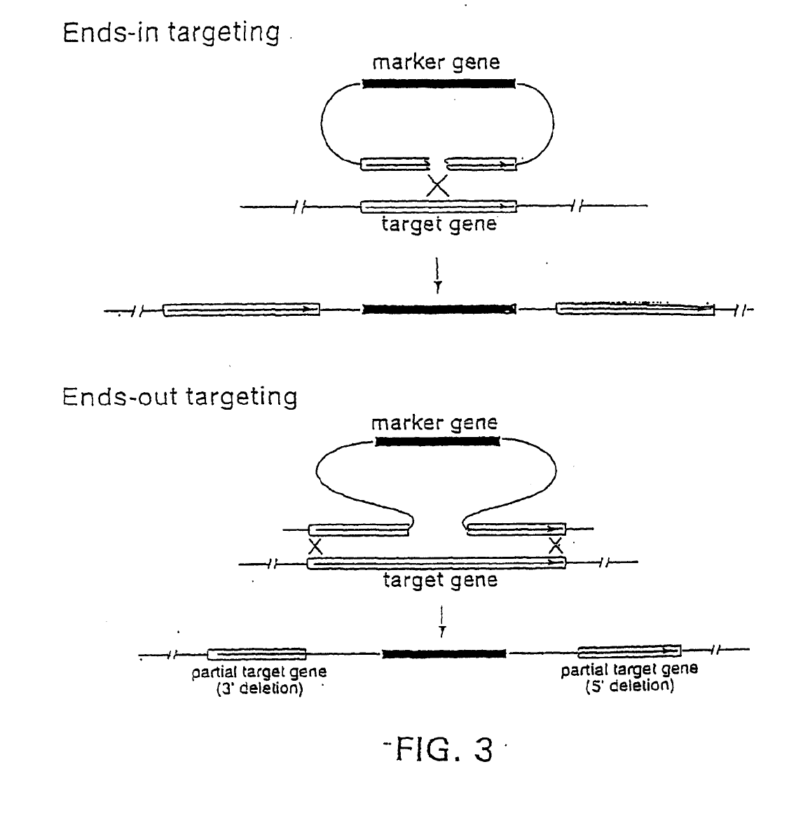

“Recombinogenic donor” is the term used herein to describe the structure of that part of the donor construct resulting from the action of the unique endonuclease and, if so designed, the recombinase. The recombinogenic donor is not integrated in the host chromosome and is characterized by having segments homologous to the target interrupted by a double-strand break for ends-in targeting, or having segments homologous to the target flanked by broken ends in the case of ends-out targeting. For example, a recombinogenic donor resulting from the action of a unique endonuclease acting on a recognition site introduced into a target gene modifying sequence could have a structure as diagramed in the lower part of FIG. 2, a linear DNA with endonuclease-cut ends which, if rejoined, would form a circular structure with the modifying sequence reconstituted. The donor construct can be designed either for ends-in targeting, which often results in an insertion into the target gene, or for ends-out targeting, which often results in replacement of a segment of the target, as shown in FIG. 3.

“Recombinase” is the term known in the art for a class of enzymes which catalyze site-specific excision and integration into and out of a host chromosome or a plasmid. At least 105 such enzymes are known and reviewed generally, with references, by Nunes-Duby, S. et al (1998) Nucleic Acids Res. 26:391-406, incorporated herein by reference. It is anticipated that novel recombinases will be discovered and can be utilized in the invention. Two well-known and widely used recombinases are Flp, isolated from yeast, and Cre from bacteriophage P1. Both enzymes have been shown to be expressible and functional in both procaryotes and eucaryotes. Site specificity of a recombinase is provided by a specific recognition sequence which is termed a recombinase target sequence herein. The recombinase target sequences for Flp and Cre are designated FRT, and lox, respectively.

The control of gene expression is accomplished by a variety of means well-known in the art. Expression of a transgene can be constitutive or regulated to be inducible or repressible by known means, typically by choosing a promoter that is responsive to a given set of conditions, e.g. presence of a given compound, or a specified substance, or change in an environmental condition such as temperature. In examples described herein, heat shock promoters were employed. Genes under heat shock promoter control are expressed in response to exposure of the organism to an elevated temperature for a period of time. The term “inducible expression” extends to any means for causing gene expression to take place under defined conditions, the choice of means and conditions being chosen on the basis of convenience and appropriateness for the host organism.

A “carrier host organism” is one that has been stably transformed to carry one or more genes for expression of a function used in the process of the invention. Functions which can be provided in a carrier host organism include, but are not limited to, unique restriction endonucleases and recombinases.

Many of the genetic constructs used herein are described in terms of the relative positions of the various genetic elements to each other. “Adjacent” is used to indicate that two genetic elements are next to one another without implying actual fusion of the two sequences. For example, two segments of DNA adjacent to one another can be separated by oligonucleotides providing a restriction site, or having no apparent function. “Flanking” is used to indicate that the same, similar, or related sequences exist on either side of a given sequence. For example, in the upper diagram of FIG. 2, the y⁺ gene is shown flanked by β2t segments. That construct is in turn flanked by FRT sites oriented parallel to one another. Segments described as “flanking” are not necessarily directly fused to the segment they flank, as there can be intervening, non-specified DNA. These and other terms used to describe relative position are used according to normal accepted usage in the field of genetics.

The method of the invention can be used for gene targeting in any organism. Minimum requirements include a method to introduce genetic material into the organism (either stable or transient transformation), existence of a unique endonuclease that can be expressed in the host organism (or a modified host organism) without harming the organism, and sequence information regarding the target gene or a DNA clone thereof. The efficiency with which homologous recombination occurs in the cells of a given host varies from one class of organisms to another. However the use of an efficient selection method or a sensitive screening method can compensate for a low rate of homologous recombination. Therefore the basic tools for practicing the invention are available to those of ordinary skill in the art for such a wide range and diversity of organisms that the successful application of such tools to any given host organism is readily predictable.

Transformation can be carried out by a variety of known techniques, depending on the organism, on characteristics of the organism's cells and of its biology. Stable transformation involves DNA entry into cells and into the cell nucleus. For single-celled organisms and organisms that can be regenerated from single cells (which includes all plants and some mammals), transformation can be carried out by in vitro culture, followed by selection for transformants and regeneration of the transformants. Methods often used for transferring DNA or RNA into cells include micro-injection, particle gun bombardment, forming DNA or RNA complexes with cationic lipids, liposomes or other carrier materials, electroporation, and incorporating transforming DNA or RNA into virus vectors. Other techniques are known in the art. For a review of the state of the art of transformation, see standard reference works such as Methods in Enzymology, Methods in Cell Biology, Molecular Biology Techniques, all published by Academic Press, Inc. New York DNA transfer into the cell nucleus occurs by cellular processes, and can sometimes be aided by choice of an appropriate vector, by including integration site sequences which can be acted upon by an intracellular transposase or recombinase. For reviews of transposase or recombinase mediated integration see, e.g., Craig, N. L K. (1988) Ann. Rev. Genet. 22:77; Cox, M. M. (1988) In Genetic Recombination (R. Kucherlapati and G. R. Smith, eds.) 429-443, American Society for Microbiology, Washington, D.C.; Hoess, R. H. et al. (1990) In Nucleic Acid and Molecular Biology (F. Eckstein and D. M. J. Lilley eds.) Vol. 4, 99-109, Springer-Verlag, Berlin. Direct transformation of multicellular organisms can often be accomplished at an embryonic stage of the organism. For example, in Drosophila, as well as other insects, DNA can be micro-injected into the embryo at a multinucleate stage where it can become integrated into many nuclei, some of which become the nuclei of germ line cells. By incorporating a marker as a component of the transforming DNA, non-chimeric progeny insects of the original transformant individual can be identified and maintained. Direct microinjection of DNA into egg or embryo cells has also been employed effectively for transforming many species. In the mouse, the existence of pluripotent embryonic stem (ES) cells that are culturable in vitro has been exploited to generate transformed mice. The ES cells can be transformed in culture, then micro-injected into mouse blastocysts, where they integrate into the developing embryo and ultimately generate germline chimeras. By interbreeding heterozygous siblings, homozygous animals carrying the desired gene can be obtained. Recently stable germline transformations were reported in mosquito (Catteruccia F., et al., (2000) Nature 405:954-962). For reviews of the methods for transforming multicellular organisms, see, e.g. Haren et al. (1999) Annu. Rev. Microbiol. 53:245-281; Reznikoff et al. (1999) Biochem. Biophys. Res. Commun. Dec.29:266(3):729-734; Ivics et al. (1999) 60:99-131; Weinberg (1998) Mar.26:8(7):R244-247; Hall et al. (1997) FEMS Microbiol. Rev. Sep:21(2):157-178; Craig (1997) Annu. Rev. Bioclem. 66:437-474; Beall et al. (1997) Genes Dev. Aug.15:11(16):2137-2151. Transformed plants are obtained by a process of transforming whole plants, or by transforming single cells or tissue samples in culture and regenerating whole plants from the transformed cells. When germ cells or seeds are transformed there is no need to regenerate whole plants, since the transformed plants can be grown directly from seed.

A transgenic plant can be produced by any means known to the art, including but not limited to Agrobacterium tumefaciens-mediated DNA transfer, preferably with a disarmed T-DNA vector, electroporation, direct DNA transfer, and particle bombardment, see e.g., Davey et al. (1989) Plant Mol. Biol. 13:275; Walden and Schell (1990) Eur. J. Biochem. 192:563; Joersbo and Burnstedt (1991) Physiol. Plant. 81:256; Potrykus (1991) Annu. Rev. Plant Physiol. Plant Mol. Biol. 42:205; Gasser and Fraley (1989) Science 244:1293; Leemans (1993) Bio/Technology. 11:522; Beck et al. (1993) Bio/Technology. 11:1524; Koziel et al. (1993) Bio/Technology. 11:194; and Vasil et al. (1993) Bio/Technology. 11:1533. Techniques are well-known to the art for the introduction of DNA into monocots as well as dicots, as are the techniques for culturing such plant tissues and regenerating those tissues. Regeneration of whole transformed plants from transformed cells or tissue has been accomplished in most plant genera, both monocots and dicots, including all agronomically important crops.

A unique endonuclease site can be a recognition site for a rare-cutting endonuclease or for any other enzyme that generates a double-stranded break in DNA at the recognition site, including, for example, a transposase. The only requirement for the invention is that the enzyme does not act elsewhere on the genome of the organism, or at a minimum, that activity of the enzyme does not reduce viability of the organism significantly.

Markers are used for a variety of purposes known in the art of genetics. A molecular marker, such as an RFLP or SSR marker can serve to indicate the presence of a given gene or DNA sequence linked to it, and can also provide location information relative to the presence of other markers. A selectable marker is a segment of genetic information, usually a gene, which, when expressed, can convey a reproductive differential or survival advantage or disadvantage to the organism possessing the marker, under environmental conditions which the investigator can control. Positive selection is provided when the marker conveys an advantage to the organism or cell possessing it, compared to those lacking it. Negative selection is provided when the marker conveys a relative disadvantage to an organism or cell possessing the marker. A selectable marker gene can be constitutive or placed under inducible expression control, so that the selection can be activated or inactivated under the control of the investigator. Positive selection can be provided, for example, by a gene conferring resistance to an antibiotic or other toxin so that in the presence of the toxin cells lacking the resistance are less viable than cells possessing the resistance. Similarly, negative selection is provided by a gene conferring sensitivity to a specific compound, so that cells possessing the gene are selectively killed in the presence of the toxin. The foregoing are merely examples of the great variety and complexity of markers used for selection, and of selection systems in general which are known in the art, and fundamental to the practice of genetics. Markers for screening are those which convey an identifiable trait (phenotype) to cells or organisms possessing the marker,- which trait is lacking in cells or organisms that do not possess the marker. An antigen not normally present in the organism or in individual cells can serve as a screening marker, using a fluorescent-tagged antibody or other tag to identify the antigen's presence. Many screening markers are known and available to those skilled in the art. The use of markers is exemplified for various aspects of the invention, however it will be understood that the manner of using markers and the choice of a particular marker type in a given situation is well-understood in the art, and that the invention does not depend on the use of any particular type of marker.

“Recombination,” in the context of the present invention, is a term for a process in which genetic material at a given locus is modified as a consequence of an interaction with other genetic material. “Homologous recombination” is recombination occurring as a consequence of interaction between segments of genetic material that are homologous, or identical, at least over a substantial length of nucleotide sequence. The minimal necessary length is functionally defined and may vary from cell to cell, or organism to organism (i.e., between species). Homologous recombination is an enzyme-catalyzed process that occurs in essentially all cell types. The reaction takes place when nucleotide strands of homologous sequence are aligned in proximity to one another and entails breaking phosphodiester bonds in the nucleotide strands and rejoining with neighboring homologous strands or with an homologous sequence on the same strand. The breaking (cutting) and rejoining (splicing) can occur with precision such that sequence fidelity is retained. Homologous recombination between a target gene and a donor construct of identical sequence except for a marker can result in reconstitution of the target, distinguishable only by the presence of the marker. Homologous recombination occurs only rarely, if ever, unless the donor and the target can be present in physical proximity to one another. In one embodiment of the invention, the donor construct is integrated at a chromosomal site that is not near the target. The cells are then provided with means for freeing the recombinogenic donor from its chromosomal locus to allow homologous recombination to take place. In another embodiment, the donor construct is present in the cell but not integrated into the chromosome, for example as an autonomously replicating plasmid or as a non-replicating, transiently present plasmid. In either of the latter cases, the donor construct is already free to approach the target and the action of rendering the donor recombinogenic by introducing a double strand DNA break stimulates homologous recombination with the target. The frequency of homologous recombination is influenced by a number of factors. Different organisms vary with respect to the amount of homologous recombination that occurs in their cells and the relative proportion of homologous to non-homologous recombination that occurs is also species-variable. The length of the donor-target region of homology affects the frequency of homologous recombination events, the longer the region of homology, the greater the frequency. The length of the homology region needed to observe homologous recombination is also species-variable. However, differences in the frequency of homologous recombination events can be offset by the sensitivity of selection for the recombinations that do occur. With sufficiently sensitive selection, e.g., by choosing a combination of positive and negative selection, virtually every recombination event can be identified. Other factors, such as the degree of homology between the donor and the target sequences will also influence the frequency of homologous recombination events, as is well-understood in the art. It will be appreciated that absolute limits for the length of the donor-target homology or for the degree of donor-target homology cannot be fixed, but depend on the number of potential events which can be scored and the sensitivity of selection. Where it is possible to screen 10⁹ events, for example, in cultured cells, a selection that can identify 1 recombination in 10⁹ cells will yield useful results. Where the organism is larger, or has a longer generation time, such that only 100 individuals can be scored in a single test, the recombination frequency must be higher and selection sensitivity is less critical. All such factors are well known in the art, and can be taken into account when adapting the invention for gene targeting in a given organism. The invention can be most readily carried out in the case of organisms which have rapid generation times or for which sensitive selection systems are available, or for organisms that are single-celled or for which pluripotent cell lines exist that can be grown in culture and which can be regenerated or incorporated into adult organisms. In the former case, the invention is demonstrated for the fruit fly, Drosophila. The latter case is demonstrated with a plant, Arabidopsis. These organisms are representative of their respective classes and the description demonstrates how the invention can be applied throughout those classes. It will be understood by those skilled in the art that the invention is operative independent of the method used to transform the organism. Further, the fact that the invention is applied to such disparate organisms as plants and insects demonstrates the widespread applicability of the invention to living organisms generally.

The organisms in which gene targeting can be accomplished according to the invention include, but are not limited to: insects, including insect species of the orders Coleoptera, Diptera, Hemiptera, Homoptera, Hymenoptera, Lepidoptera and Ortiloptera; plants, including both monocotyledonous plants (monocots) including, but not limited to, maize, rice, wheat, oats and other grain crops, and dicotyledonous plants (dicots) including, but not limited to, potato, soybean and other legumes, tomato, members of the Brassica family, Arabidopsis, tobacco, grape and ornamental species such as roses, carnations, orchids and the like; mammals, including known transformable species such as mouse, rat, sheep, and pig, and others, as transformation methods are developed, including bovine and primates including humans; birds, including food species such as chicken, turkey, duck and goose; fish, including species raised for food or sport including trout, salmon, catfish, tilapia, ornamental breeds such as koi and goldfish, and the like; and shellfish, including oyster, clam, shrimp and the like. Gene targeting in such organisms is useful to accomplish genetic modification to impart disease resistance, improve hardiness and vigor, remove genetic defects, improve product quality or yield, impart new desirable traits, alter growth rates or in the case of pest species and disease vectors, introduce, alter or remove genes affecting the ability of the pest or vector to spread disease or cause damage.

It will be understood that the invention is also useful for gene targeting in somatic cells and tissues, and is not limited to germ line or pluripotent cells. Targeting in somatic cells provides the ability to make desired and specific genetic modification to target host cells and tissues. Targeting in somatic cells now provides a means of producing transgenic animals through the nuclear transfer technique (McCreath, K. J. et al. (2000) Nature 405:1066-1069; Polejaeva, I. A. et al., (2000) Nature 407:86-90). Transformation methods using tissue or cell-type-specific vectors can be employed for providing a desired donor construct in the cells of choice, or the cells can be transformed by non-specific means, using tissue-specific promoters to ensure activation of targeting the cells of choice. Obvious choices include tumor cells and specific tissues affected by a genetic defect. The methods of the invention are therefore useful to expand and supplement the available techniques of gene therapy.

A factor which influences targeting efficiency is the extent of homology or nonhomology between donor and target. There are many reports showing that increased donor:target homology increases the absolute targeting frequency in mammalian cells, see e.g., M. J. Shulman et al. (1990) Mol. Cell. Biol. 10:466, C. Deng, M. R. Capecchi (1992) Mol. Cell. Biol. 12:3365. In Drosophila, investigators have examined the effect of homology in the context of P transposon break-induced gene conversion. The ds break that is left behind when a P element transposes is a substrate for gene conversion, and may use ectopically-located homologous sequences as a template. Dray and Gloor (, J. B. Scheeber, G. M. Adair (1994) Mol. Cell. Biol. 14:6663; T Dray, H G. B. Gloor (1997) Genetics 147:684) found that as little as 3 kb of total template:target homology sufficed to copy a large non-homology segment of DNA into the target with reasonable efficiency. In prior work on FLP-mediated DNA mobilization, very different efficiencies were observed for FLP-mediated integration at a target FRT when comparing experiments in which the donor and target shared different extents of homology (M. M. Golic (1997) Nucleic Acid Res. 25:3665). Integration was approximately 10-fold more efficient when the donor and target shared 4.1 kb of homology than when they shared only 1.1 kb of homology, suggesting the possibility that interactions between an extrachromosomal DNA molecule and a chromosomal sequence may be stabilized to some degree by shared sequences. If the extent of homology is an important factor, increasing the extent of donor:target homology may increase the overall frequency of targeting, and as a consequence provide a means to shift the ratio of targeted to non-targeted events. The limited data available from Drosophila leads us to conclude that 2-4 kb of donor:target homology is sufficient for efficient targeting, although in the experiment of Example 2 the donor and target shared 8 kb of homology.

The gene targeting technique of the invention is efficient enough that chemical or genetic selection methods were not needed for the described embodiment but these can be implemented as part of the scheme if desired. Furthermore, the procedure in general does not require special lines of cultured cells, as does mouse gene targeting. Because the technique can be carried out in the intact organism it can be used for gene targeting in many other species of animals and plants, with the only requirement being that a method of transformation exist.

It will be understood that for each of the specific features of the process of the invention as just described there exists a panoply of functional equivalents which can be employed, as desired and as appropriate, to carry out the invention.

Use of Other Site-specific Recombinases and/or Site-specific Endonucleases.

There are a large number of site-specific endonucleases known that function similarly to FLP, and that can be substituted in this procedure. For example the Cre recombinase and its lox target site can be employed instead of the FLP-FRT system. Many other site-specific endonucleases are listed by Nunes-Duby et al (1998) Nucleic Acids Research 26:391406, and there are no doubt many yet to be found.

The I-SceI intron-homing endonuclease is also one of a large number of functionally similar rare-cutting endonucleases. Many of these, for instance I-TliI, I-CeuI, I-CreI, I-PpoI and PI-PspI, can be substituted for I-SceI in the targeting scheme. Many are listed by Belfort and Roberts (1997) Nucleic Acids Research 25:3379-3388). Many of these endonucleases derive from organelle genomes in which the codon usage differs from the standard nuclear codon usage. To use such genes for nuclear expression of their endonucleases it may be necessary to alter the coding sequence to match that of nuclear genes. This can be done by synthesizing the gene as a series of oligonucleotides, that are then ligated together in the proper order to produce a segment of DNA that encodes the entire endonuclease with nuclear codon usage.

Introduction of mutations.

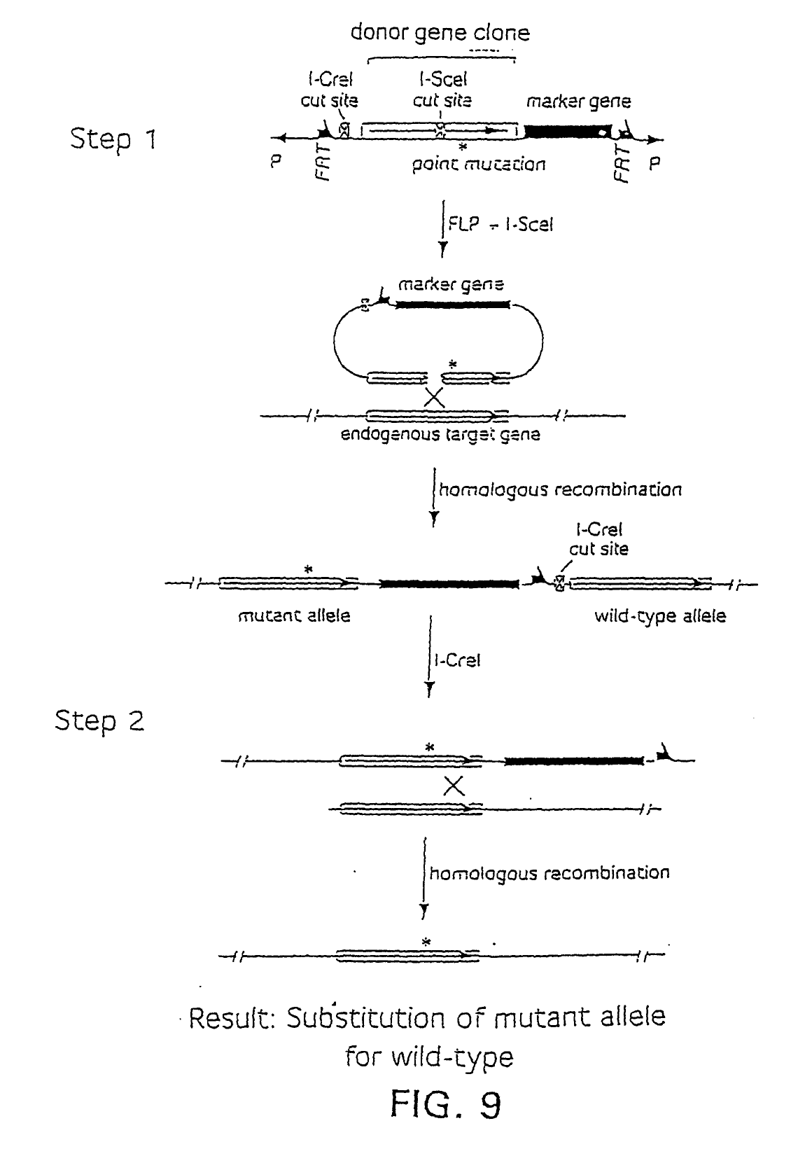

The gene targeting technique described herein can be used to substitute one allele for another at the targeted locus. This provides a way to insert large or small mutations into a targeted locus, or to convert a mutant allele into the wild-type allele. In cases where the mutant phenotype of the targeted gene is unknown, molecular techniques, such as PCR, can be used to detect the mutated allele. A two-step method that provides a simple genetic method to detect allelic substitutions can also be used (FIG. 9).

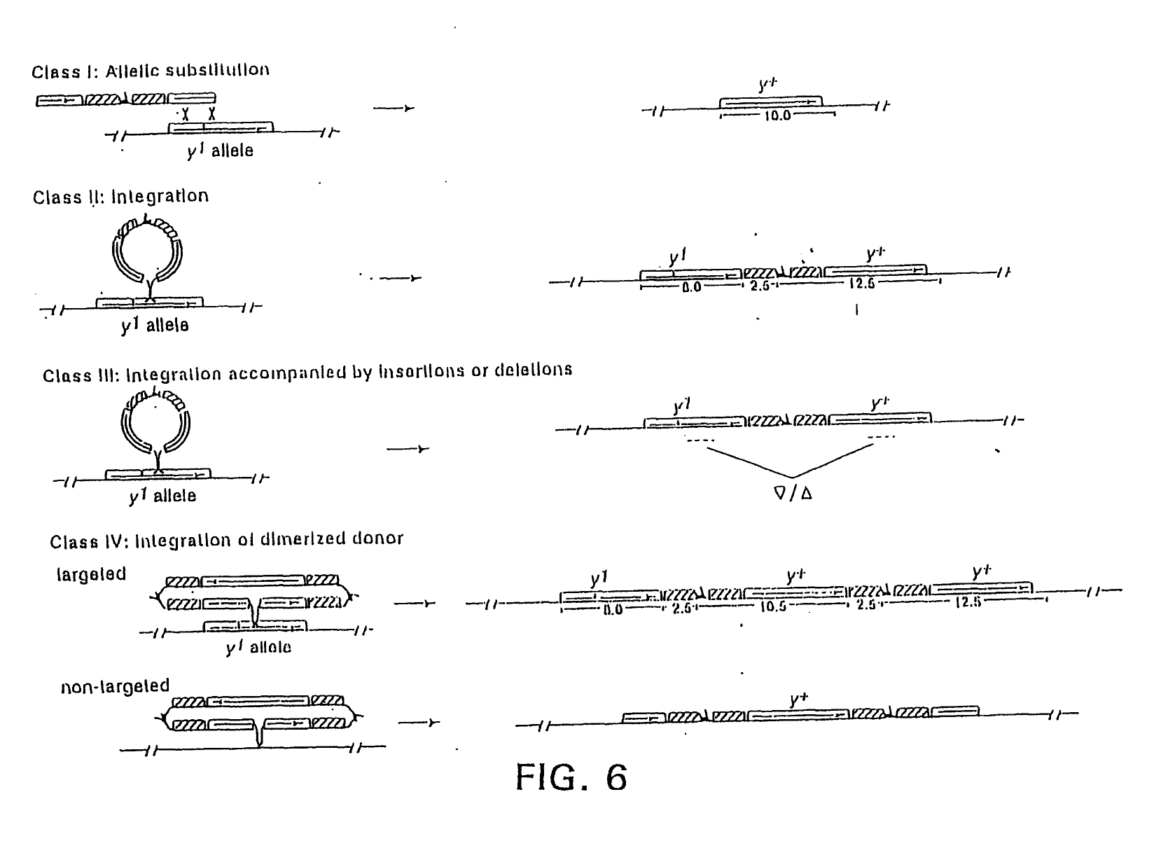

To make a donor construct, a cloned copy (or partial copy) of the target gene is engineered to carry the desired mutation and an I-SceI cut site. In this example a simple point mutation is introduced, for instance a change of a coding codon to a stop codon. This technique is not limited to point mutations; insertions or deletions of varying sizes can be introduced also. The introduced mutation may be placed to the left or right of the I-SceI recognition site; in FIG. 9 it is shown to the right for illustrative purposes only. The donor version of the target is placed into a transposon vector between FRTs, along with a marker gene (such as the white+eye color gene), and a cut site for a second site-specific endonuclease (such as I-CreI), and transformed into Drosophila. The engineered mutation is then recombined into the target gene as a Class II (FIG. 6) targeting event by simply screening for altered chromosomal linkage of the marker gene. The product is a tandem duplication with a point mutation in one copy, and the marker gene and I-CreI cut site between the tandem copies of the target gene. Molecular analysis is used to confirm the presence of the introduced mutation.

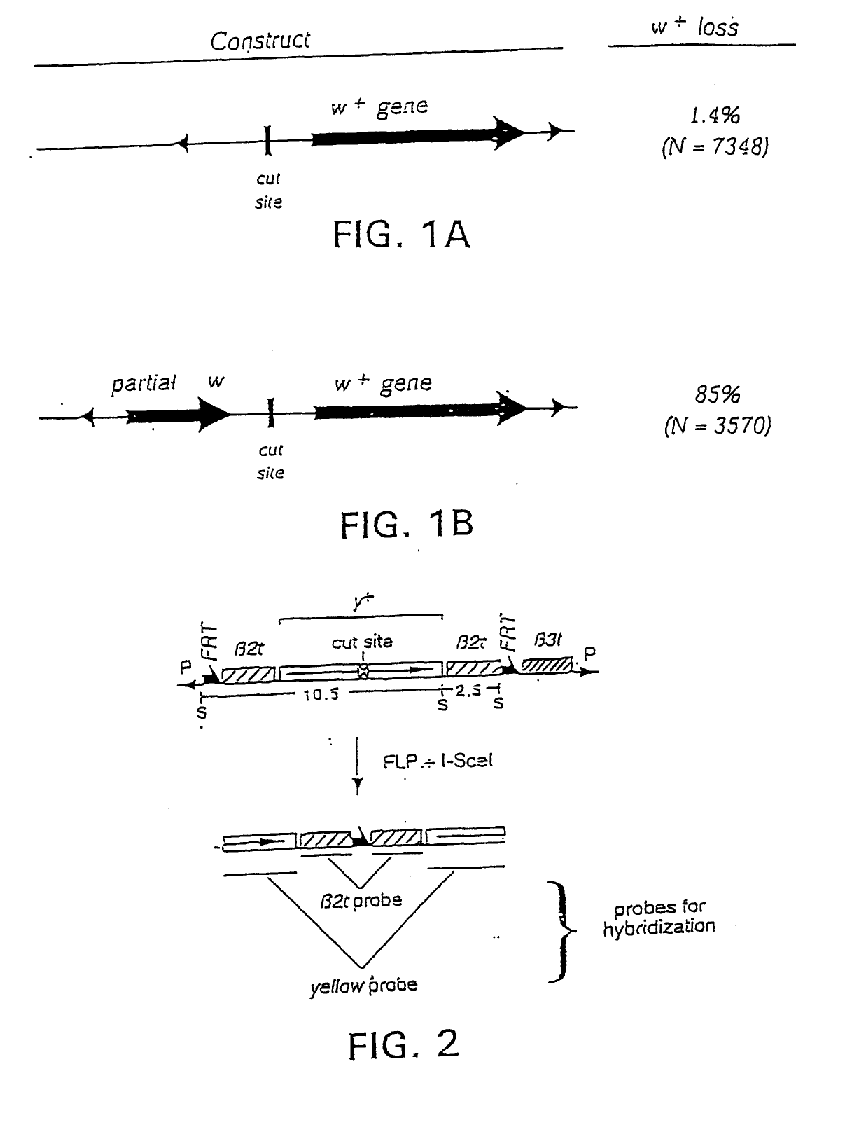

In the second step, I-CreI endonuclease is introduced into the flies produced in step 1 (using a transgene or any of several other methods discussed here). This endonuclease cuts the chromosomes in the region between the tandem repeats, causing frequent reduction of the two tandem copies to a single copy by recombination (as shown by the data of FIG. 1). Loss of the tandem repeat is easily recognized because the w⁺ marker gene is lost in the process. In a fraction of the cases, the crossover that eliminates the tandem duplication will occur to the right of the point mutation, and the resultant allele carries the introduced mutation. Molecular or genetic analysis can be used to determine which of the marker-loss alleles carry the mutation, using methods and markers known to those skilled in the art.

The foregoing two step method requires no knowledge of the mutant phenotype. It is based simply on the segregation and then loss of a marker gene. A variation of the foregoing procedure is to introduce two point mutations into the donor copy of the gene: one on each side of the I-SceI cut site. In this case, the two alleles of the target gene in the tandem duplication would each be mutated. Molecular analysis is used to confirm the presence of both point mutations. Step 2, as described, is not be necessary in order to generate a mutant organism. Moreover, because a marker gene is present between the mutant alleles, it is very easy to follow the segregation of the mutant locus through crosses.

This procedure can also provide a way to select for the survival of the mutant organisms. For instance, if the marker gene was a chemical resistance gene, then treatment of the organisms with the chemical selects for those carrying the tandem duplication, and the engineered alleles.

If desired, step 2 can be implemented to reduce the two mutant alleles to a single mutant allele. Only crossovers that occurred between the two mutations would restore the wild-type; all others produce an allele carrying one or the other mutation.

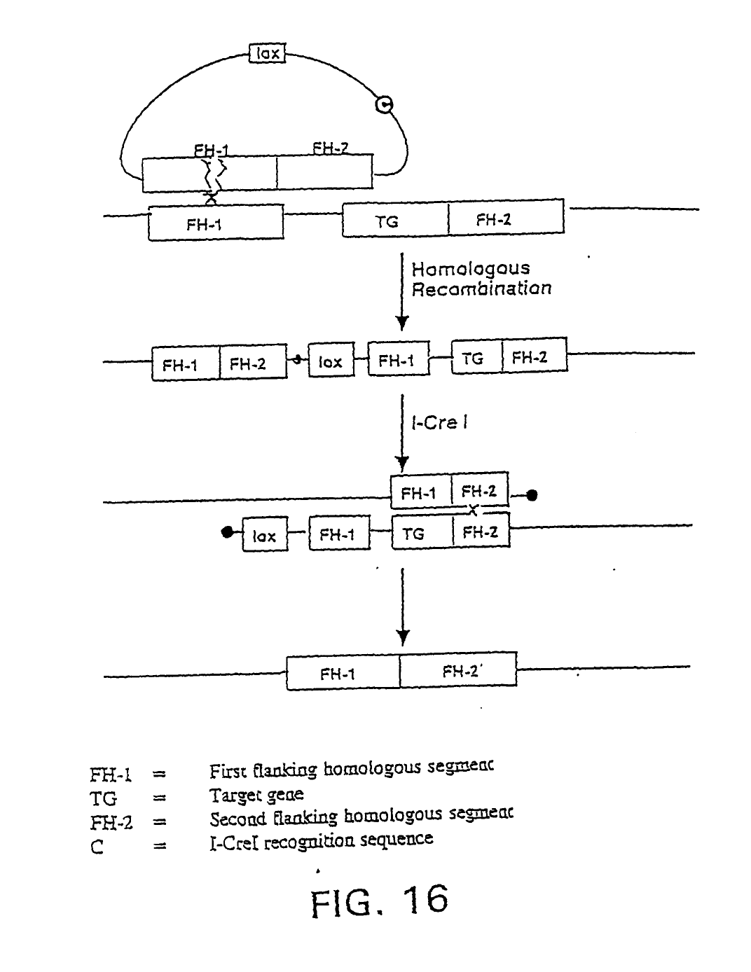

A two-step process can be employed for generating a null mutation of a target gene. Two homologous recombinations are targeted for flanking homologous segments on either side of the target gene resulting in a deletion of the target gene, as diagramed in FIG. 16. The donor construct includes a first flanking homologous segment carrying a unique endonuclease site, such as I-SceI, a second flanking homologous segment, a recombinase gene, such as I-CreI and a recombinase recognition site, such as lox. In the target genome, the target gene lies between the two flanking homologous segments. A double strand break induced in the donor by I-SceI endonuclease stimulates homologous recombination in the first flanking homologous segment which integrates the donor construct into the genome as shown in the first step of FIG. 16. Induction of I-CreI results in a cleavage at its recognition site to allow pairing and recombination within the second flanking homologous segment, as shown in the second step of FIG. 16. The effect of the second recombination event is deletion of the target gene and retention of the flanking homologous segments, as shown in the bottom line of FIG. 16. Appropriate selection markers can be incorporated to identify stages of the process. Deletion of the target can, itself, serve as a selectable event, depending on the null phenotype. Other techniques of deletion targeting or replacement targeting can be employed, as known in the art, for example, by employing an ends-out targeting construct.

Targeting by Use of a Site-specific Endonuclease Only.

Donor constructs can also be engineered to contain two unique endonuclease cut sites such as I-SceI sites that flank a cloned donor version of the target locus and a marker gene. The cloned donor could be engineered in two halves so that the right half of the donor version of the target gene is located at the left end of the construct and vice-versa, with the marker gene between the halves. After introducing such a construct into the organism, double cutting at the flanking sites releases a donor molecule that is essentially identical to the released donor molecule shown in the lower half of FIG. 2.

Ends-out Targeting.

Ends-out targeting can also be applied using a site-specific recombinase and unique endonuclease to release the donor molecule, or using only a unique site-specific endonuclease, but including two sites for site-specific endonuclease cutting within the donor construct. A donor construct intended for ends-out targeting is prepared by providing that the coding sequences of segment lying on either side of the inserted endonuclease site are in antiparallel orientation with respect to one another. Where the normal coding sequence of the target is abcdefgh, insertion of an endonuclease site between d and e provides abcd/efgh, where the two parts separated by the cleavage site are in parallel orientation. Cleavage yields dcba-hgfe which can recombine by “ends-in” recombination. For ends-out targeting the antiparallel orientation is constructed, dcba/hgfe, which upon cleavage yields abcd-efgh. See FIG. 3.

Other ends-out targeting schemes are within the scope of the invention. Such schemes can involve the incorporation of a negatively selectable marker at a site which can be used to favor targeted over non-targeted insertions or at a site which can be used to eliminate progeny with the donor chromosome.

Use in Other Insects.

The method of the invention can be applied to other insects also. For a review of genetic manipulations in insects see Insect Transgenesis Methods and Applications, Handler, A. M., and A. A. James eds. (2000) CRC Press, Boca Raton, Fla., which is incorporated by reference in its entirety. One potential problem in other insects is a paucity of genetic markers that can be followed to do the segregation screening. This paucity of markers applies to many other organisms in which the invention can be used for gene targeting. The problem can be dealt with by placing two dominant markers in the donor transgene. One of the markers (for instance a green fluorescent protein [GFP] gene) would be placed outside the FRTs. The second marker (for instance a chemical resistance gene) would be placed between the FRTs along with the target locus. After freeing the donor construct the first marker will stay in place, while the second marker will accompany the donor targeting DNA to the targeted locus. Therefore, after induction of FLP and I-SceI enzymes, screening can be carried out by looking for animals that are resistant to the chemical, but which do not show GFP fluorescence. These would be individuals in which the resistance gene had segregated from the GFP donor chromosome marker gene. Targeting can be verified by molecular means. A positive-negative selection method can also be employed in such a screen to increase the sensitivity of recombinant detection.

Use in Other Animals.

This method can also be applied in other animals, including, but not limited to, mice, humans, cattle, sheep, pigs, nematodes, amphibians, and fish.

Use in Plants.

Targeted alteration of plant genomes can be carried out using the procedures described herein.

It is contemplated that the gene targeting methods of the invention can be used in a variety of plants such as grasses, legumes, starchy staples, Brassica family members, herbs and spices, oil crops, ornamentals, woods and fibers, fruits, medicinal plants, and alternative and other crops. Preferably the invention can be used in plants such as sugar cane, wheat, rice, maize, potato, sugar beet, cassava, barley, soybean, sweet potato, oil palm fruit, tomato, sorghum, orange, grape, banana, apple, cabbage, watermelon, coconut, onion, cottonseed, rapeseed, and yam.

Grasses include, but are not limited to, wheat, maize, rice, rye, triticale, oats, barley, sorghum, millets, sugar cane, lawn grasses, and forage grasses. Forage grasses include, but are not limited to, Kentucky bluegrass, timothy grass, fescues, big bluestem, little bluestem and blue gamma.

Legumes include, but are not limited to, beans like soybean, broad or windsor bean, kidney bean, lima bean, pinto bean, navy bean, wax bean, green bean, butter bean, and mung bean; peas like green pea, split pea, black-eyed pea, chick-pea, lentils, and snow pea; peanuts; other legumes like carob, fenugreek, kudzu, indigo, licorice, mesquite, copaifera, rosewood, rosary pea, senna pods, tamarind, and tuba-root; and forage crops like alfalfa.

Starchy staples include, but are not limited to, potatoes of any species including white potato, sweet potato, cassava, and yams.

Brassica, include, but are not limited to, cabbage, broccoli, cauliflower, brussels sprouts, turnips, and radishes.

Alternative and other crops include, but are not limited to, quinoa, amaranth, tarwi, tamarillo, oca, coffee, tea, and cacao.

Herbs and spices include, but are not limited to, cinnamon, black and white pepper, cloves, nutmeg and mace, ginger and turmeric, saffron, hot chilies and other capsicum peppers, vanilla, allspice, mint, parsley family herbs (e.g., parsley, dill, caraway, fennel, celery, anise, coriander, cilantro, cumin, chervil) mustard family members (e.g., mustard and horseradish), and lily family members (e.g., onion, garlic, leeks, shallots, and chives).

Oil crops include, but are not limited to, soybean, palm, rapeseed, sunflower, peanut, cottonseed, coconut, olive palm kernel.

Woods and fibers include, but are not limited to, cotton, flax, and bamboo.

Both site-specific recombinases [Dale and Ow, (1991) PNAS 88:10558-10562L Lyznik et al., (1996) Nucleic Acids Res. 24(19)3784-3789]; and site-specific unique endonucleases [Puchta et al. (1996) PNAS 93:5055-5060] have been shown to function in plants. The two can be used combinatorially to bring about gene targeting in plants.

Lloyd and Davis (1994) Mol. Gen. Genetics 242:653-657 demonstrated that the cauliflower mosaic virus (CMV) 35S promoter and terminator can be used to direct expression of FLP in tobacco plants. Puchta et al. demonstrated the same method for expression of the I-SceI endonuclease in tobacco. In other examples, recombinases have also been expressed in plants using heat-shock promoters [Kilby et al., (1995) The Plant J. 8:637-652; Sieburti et al., (1998) Development 125:4303-4312]. Transformation of plants was accomplished by use of Agrobacterium T-DNA in those cases. Similar methodology can be used in other plants, or transformation of tissues of cultured cells may be accomplished by biolistic DNA-coated particle bombardment.

Functional recombinase and/or endonuclease activity may be achieved by transgene expression, by introduction of appropriate synthetic mRNAs, or introduction of the protein themselves.

Essentially the entire panoply of unique endonucleases, recombinases and marker genes can be expressed in plants as constitutive, developmental stage-specific, or inducible transgenes. A variety of known inducible promoters that function in plants are available to those skilled in the art, including heat shock promoters. Development stage-specific promoters are useful, for example where it is advantageous to carry out targeting in specific cell types or at specific times of development; for example, during embryo development, within the cells of shoot apical meristem, or in mother cells that undergo meisosis. A number of such promoters are known; e.g., the NZZ promoter [Schiefthaler, et al. (1999) Proc. Natl. Acad. Sci. USA 96:11664-11669]; SPL [Yang -et al (1999) Genes and Development 13:2108-2117]; DIF1 [Bhatt et al (1999) Plant J. 19:463-472]; SYN1 [Bai et al (1999) Plant Cell 11:417-430]; ASK1 [Yang et al. (1999) Proc. Natl. Acad. Sci. USA 96:11416-11421]; AtDMC1 [Klimyuk and Jones (1997) Plant J. 11:1-14].

Techniques and agents for introducing and selecting for the presence of heterologous DNA in plant cells and/or tissue are well-known. Selection can be positive or negative. Genetic markers allowing for the selection of heterologous DNA in plant cells are well-known, e.g., genes carrying resistance to an antibiotic such as kanamycin, hygromycin, gentamycin, or bleomycin. The marker allows for selection of successfully transformed plant cells growing in the medium containing the appropriate antibiotic because they will carry the corresponding resistance gene. In most cases the heterologous DNA which is inserted into plant cells contains a gene which encodes a selectable marker such as an antibiotic resistance marker, but this is not mandatory. An exemplary drug resistance marker is the gene whose expression results in kanamycin resistance, i.e., the chimeric gene containing nopaline synthetase promoter, Tn5 neomycin phosphotransferase II and nopaline synthetase 3′ non-translated region described by Rogers et al., Methods for Plant Molecular Biology, A. Weissbach and H. Weissbach, eds., Academic Press, Inc., San Diego, Ca. (1988). Negative selectable markers which can be used in the invention include, but are not limited to, coda [Stougaard (1993) Plant Journal 3:755-761] tms2 [Depicker et al., (1988) Plant Cell Rep. 7:63-66] nitrate reductase [Nussame et al., (1991) Plant Journal 1:267-274] and SU1 [O'keef et al. (1994) Plant Physiol. 105:473-482].

Techniques for genetically engineering plant cells and/or tissue with an expression cassette comprising an inducible promoter or chimeric promoter fused to a heterologous coding sequence and a transcription termination sequence are to be introduced into the plant cell or tissue by Agrobacterium-mediated transformation, electroporation, microinjection, particle bombardment or other techniques known to the art. The expression cassette advantageously further contains a marker allowing selection of the heterologous DNA in the plant cell, e.g., a gene carrying resistance to an antibiotic such as kanamycin, hygromycin, gentamycin, or bleomycin. Assays for phenolic acid esterase and/or xylanase enzyme production are taught herein or in U.S. Pat. No. 5,824,533, for example, and other assays are available to the art.

A DNA construct carrying a plant-expressible gene or other DNA of interest can be inserted into the genome of a plant by any suitable method. Such methods may involve, for example, the use of liposomes, electroporation, diffusion, particle bombardment, microinjection, gene gun, chemicals that increase free DNA uptake, e.g., calcium phosphate coprecipitation, viral vectors, and other techniques practiced in the art. Suitable plant transformation vectors include those derived from a Ti plasmid of Agrobacterium tumefaciens, such as those disclosed by Herrera-Estrella (1983), Bevan (1983), Klee (1985) and EPO publication 120,516 (Schilperoort et al.). In addition to plant transformation vectors derived from the Ti or root-inducing (Ri) plasmids of Agrobacterium, alternative methods can be used to insert the DNA constructs of this invention into plant cells.

The choice of vector in which the DNA of interest is operatively linked depends directly, as is well known in the art, on the functional properties desired, e.g., replication, protein expression, and the host cell to be transformed, these being limitations inherent in the art of constructing recombinant DNA molecules. The vector desirably includes a prokaryotic replicon, i.e., a DNA sequence having the ability to direct autonomous replication and maintenance of the recombinant DNA molecule extra-chromosomally when introduced into a prokaryotic host cell, such as a bacterial host cell. Such replicons are well known in the art. In addition, preferred embodiments that include a prokaryotic replicon also include a gene whose expression confers a selective advantage, such as a drug resistance, to the bacterial host cell when introduced into those transformed cells. Typical bacterial drug resistance genes are those that confer resistance to ampicillin or tetracycline, among other selective agents. The neomycin phosphotransferase gene has the advantage that it is expressed in eukaryotic as well as prokaryotic cells.

Typical expression vectors capable of expressing a recombinant nucleic acid sequence in plant cells and capable of directing stable integration within the host plant cell include vectors derived from the tumor-inducing (Ti) plasmid of Agrobacterium tuinefaciens described by Rogers et al. (1987) Meth. in Enzymol. 153:253-277, and several other expression vector systems known to function in plants. See for example, Verma et al., No. WO87/00551; Cocking and Davey (1987) Science 236:1259-1262.

A transgenic plant can be produced by any means known to the art, including but not limited to Agrobacterium tumefaciens-mediated DNA transfer, preferably with a disarmed T-DNA vector, electroporation, direct DNA transfer, and particle bombardment [see Davey et al. (1989) Plant Mol. Biol. 13:275; Walden and Schell (1990) Eur. J. Biochem. 192:563; Joersbo and Burnstedt (1991) Physiol. Plant. 81:256; Potrykus (1991) Annu. Rev. Plant Physiol. Plant Mol. Biol. 42:205; Gasser and Fraley (1989) Science 244:1293; Leemans (1993) Bio/Technology. 11:522; Beck et al. (1993) Bio/Technology. 11:1524; Koziel et al. (1993) Bio/Technology. 11:194; and Vasil et al. (1993) Bio/Technology. 11:1533). Techniques are well-known to the art for the introduction of DNA into monocots as well as dicots, as are the techniques for culturing such plant tissues and regenerating those tissues.

Many of the procedures useful for practicing the present invention, whether or not described herein in detail, are well known to those skilled in the art of plant molecular biology. Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzmol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; and Setlow and Hollaender (1979) Genetic Engineering: Principles and Methods, Vols. 1-4, Plenum Press, New York, Kaufman (1987) in Genetic Engineering Principles and Methods, J. K. Setlow, ed., Plenum Press, New York, pp. 155-198; Fitchen et al. (1993) Annu. Rev. Microbiol. 47:739-764; Tolstoshev et al. (1993) in Genomic Research in Molecular Medicine and Virology, Academic Press. Abbreviations and nomenclature, where employed, are deemed standard in the field and commonly used in professional journals such as those cited herein.

By crossing, a plant that carries a site-specific recombinase and a unique site-specific endonuclease transgenes, under control of the same promoter, can be constructed. Alternatively, both transgenes could be placed within the same T-DNA (or other) transformation construct, and transformants selected by expression of a linked resistance gene, such as hygromycin resistance, techniques which are well-known in the art. Although the representative embodiment described below refers to transformation by using T-DNA it will be understood that other transformation methods are available to those skilled in the art, for those plant species, notably monocots, that are less amenable to T-DNA transformation.

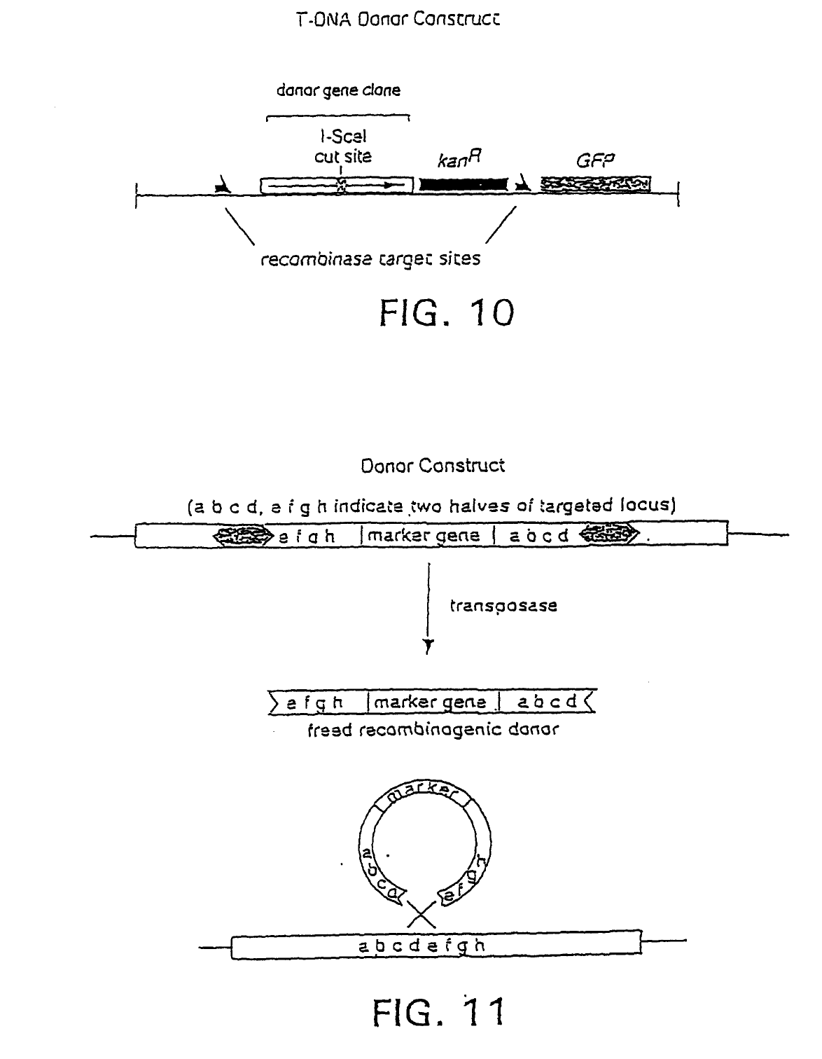

A donor construct can be constructed as diagramed in FIG. 10. The construct carries a chemical resistance gene between recombinase target sites, for instance a kanamycin resistance gene as used by Lloyd and Davis. A cloned copy of the target gene with a site-specific unique endonuclease cut site within it is also placed between the recombinase target sites. The donor construct carries a second marker gene, for instance GFP (green fluorescent protein) or GUS (beta-glucuronidase), outside of the recombinase target sites. Alternatively, the second marker gene can be a negatively-selectable marker gene such as codA, tms2, nitrate reductase, or SU1.

By crossing, a plant is generated that expresses the site-specific recombinase and site-specific endonuclease that carries the donor construct. Expression of the enzymes will cause excision and cutting of the donor molecule, which can then integrate at the target locus by homologous recombination. Recombination events can be found by screening for offspring that are kanamycin-resistant and are GFP⁻, GUS⁻, or NSM⁻ (negative selectable marker minus). In these offspring, that portion of the donor that is flanked by recombinase target sites has segregated away from the chromosome that originally carried that donor construct. Some fraction of these will be targeted recombinants, and they can be found by a molecular or genetic screen. Alternatively, it is contemplated that the donor construct, the site-specific recombinase, and site-specific endonuclease are all within the same T-DNA, obviating the need for crosses.

Because transforming DNA may undergo rearrangement in plants, it may be necessary to test several independently integrated donor constructs to find one that is suitable for use in this scheme. The main concern is that the donor T-DNA may be rearranged in such a way that the site-specific recombinase target sites flank the GFP marker, allowing for GFP loss from the chromosome that originally carried the donor construct. That occurrence would negate the screen for segregation of kan-R and GFP. Such rearranged donor constructs can be eliminated from use by molecular characterization and by testing the integrated construct with the recombinase alone. With a suitable donor insertion, the action of recombinase causes loss of kan-R but not GFP.

Use in Cultured Tissues, Cells, Nuclei, or Gametes.

The method of the invention can also be applied in cultured cells or tissues, including those cells, tissues or nuclei that can be used to regenerate an intact organism, or in gametes such as eggs or sperm in varying stages of their development.

It was demonstrated that an extrachromosomal DNA molecule with cut or broken ends that is generated in vivo, through the action of a site-specific recombinase (such as FLP) and site-specific endonuclease (such as I-SceI), is recombinogenic and can be employed for gene targeting. Alternatives for the representative embodiments described above are numerous, and not limited to the enzymes and constructs used to explain how the invention works.

Transposases can be used to generate the double-strand (ds) break, substituting for the unique endonuclease, or to carry out the excision reaction, substituting for the recombinase. Many transposons, such as P elements in Drosophila, leave behind a ds break in DNA when they transpose. This property can be used to generate broken-ended extrachromosomal molecules for targeting. Examples are indicated below, but other possibilities also exist. These examples can be carried out using stably integrated transgene constructs as the source of the donor molecule (for instance, by placing the P element construct of Example 1 into a Mariner transposon and generating stably transformed Drosophila), or transient transgenes (for instance, the T-DNA example of Method 4 below). Transposase expression can occur by expression of endogenous transposons or variants thereof, by regulated or constitutive expression from engineered gene constructs that express transposase, by use of mRNA that encodes transposase, or by using the purified transposase protein. In plants, it may be advantageous to express the transposase and/or recombinase and/or site-specific endonuclease in the megaspore and microspore mother cells, just before or during meiosis. The freed DNA fragments can be designed for ends-in targeting (as shown in the Figures) or ends-out targeting. Genetic screening, selective methods, or molecular methods, can be used to recover the targeted recombinants.

Method 1: Using Two Copies of a Transposon (FIG. 11).

A transgenic construct can be produced that carries two copies of a transposon (in this case, the P element of Drosophila) that flank the donor DNA. Recombinogenic donor DNA refers to the piece of DNA that is freed from the targeting construct as a broken-ended DNA molecule, and that is designed to cause homology-directed changes in a specific chromosomal locus. The transposition of the two transposons simultaneously, will leave behind two ds breaks that flank the intervening DNA, freeing that fragment of DNA to recombine with the chromosome at the target site.

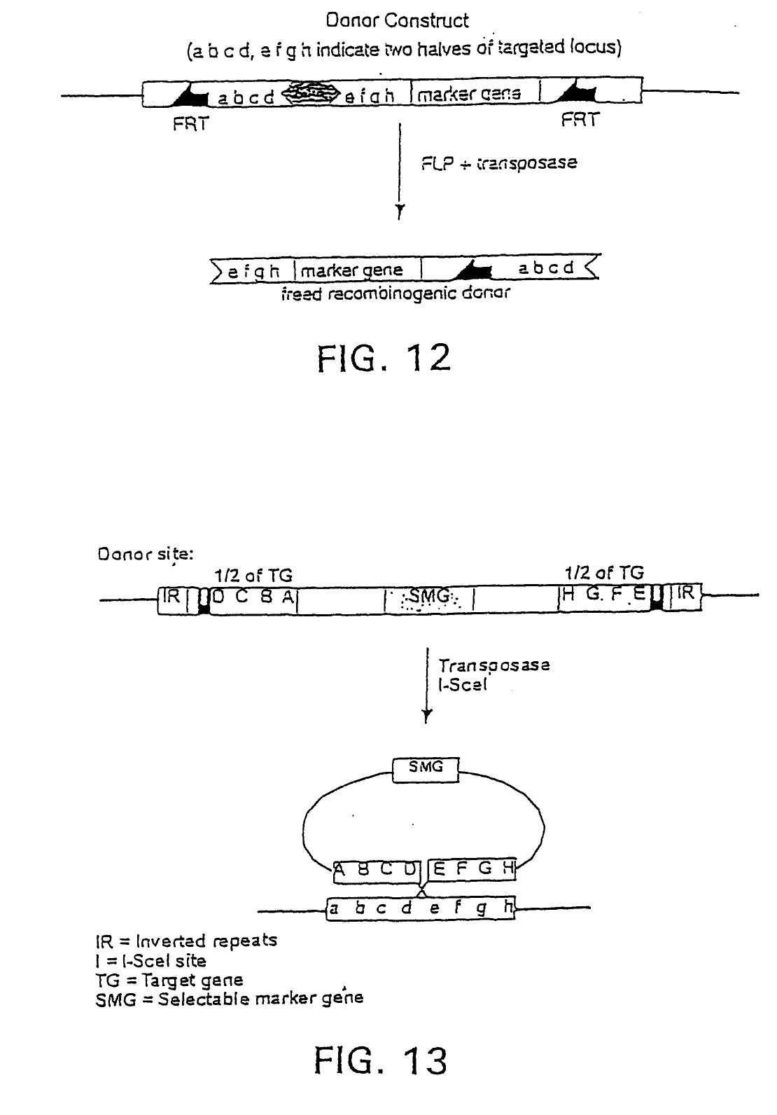

Method 2: Using a Site-Specific Recombinase and a Transposase (FIG. 12).

In this variation, a site-specific recombinase, such as FLP or Cre (or others known in the art), is used to free a segment of DNA that is flanked by recombinase recognition sites (such as FRTs or lox sites) from the donor construct. This freed DNA is circular in form. It will be converted to a linear form by transposition of a transposon from the circle, leaving behind a ds break. The procedure can be simplified by using a transient or stable circular plasmid as the donor construct. Transposition of the transposon will leave a ds break behind in the plasmid. The plasmid is then recombinogenic and can be used for targeting, but with the disadvantage that vector sequences will be included in the donor DNA. However, these can be removed through the use of site-specific recombination or homologous recombination induced by a site-specific endonuclease.

Method 3: Use of Transposons to Free DNA from the Chromosome, and a Site-Specific Endonuclease to Free a Donor from the Transposon (FIG. 13).

A transposase can be used as an alternative to a recombinase to excise the donor construct from the donor site. For ends-in targeting, the donor gene construct can be split as shown in FIG. 13 and placed within the transposon. Using a transposase for excision, the transposase and I-SceI (or other unique endonuclease) can be expressed at approximately the same time. The fundamental concept relies on the excising of the transposon at the inverted repeats by the transposase, followed by cutting at the I-SceI sites with I-SceI. The combined action of the two enzymes creates a recombinogenic donor and is similar to what can be accomplished with a site-specific recombinase and site-specific endonuclease.

Method 4: Use of T-DNA.

A method similar to that described in method 3 can be employed with T-DNA. The construct for this method is analogous to that of method 3, except for the substitution of the respective T-DNA borders for the inverted repeats. This method relies on I-SceI (or other unique endonucleases) being expressed in the transformed cells (for example, the egg cell in Arabidopsis). The idea is that in cells undergoing transformation, the T-DNA is cut by I-SceI, creating a recombinogenic donor as shown in FIG. 13.

Further explanation of the invention will be described by examination of various embodiments of the invention and reviewing various alternative means by which the invention can be carried out.

### EXAMPLE 1

The first-described embodiment of the invention was carried out in Drosophila using broken-ended extrachromosomal DNA molecules to produce homology-directed changes in a target locus. Two transgenic enzymes were used for this purpose: the FLP site-specific recombinase and the I-SceI site-specific endonuclease. FLP recombinase efficiently catalyzes recombination between copies of the FLP recombination Target (FRT) that have been placed in the genome [Golic and Lindquist (1989) Cell 59:499]. When FRTs are in the same relative orientation within a chromosome FLP excises the intervening DNA donor construct from the chromosome in the form of a closed circle. If the FRTs are close to one another this excision is nearly 100% efficient. In accord with the principles of the invention, the excised DNA donor construct molecules become recombinogenic if they carry a ds break. To generate this break we provided for a host organism in which the I-SceI intron-homing endonuclease from yeast was introduced into Drosophila. I-SceI recognizes and cuts a specific 18 bp recognition site sequence [Colleaux, L. et al. (1986) Cell 44:521; Colleaux, L. et al. (1988) Proc. Natl. Acad. Sci. USA 85:6022] which is not normally present in the Drosophila genome.

Inducible ds Breakage.

To express I-SceI in flies we constructed a heat-inducible I-SceI gene (70I-SceI) and used standard P element transformation to generate fly lines carrying the transgene. We used two chromosomally-integrated tester constructs to assay the efficacy of 70I-SceI. Each carried a white⁺ (w⁺) reporter gene with an I-SceI cut site adjacent to it as described herein. One of the tester constructs also carried a partial duplication of the white reporter gene (FIG. 1). To test for cutting at I-SceI recognition sites, flies that carried 70I-SceI and a reporter construct were generated by crossing, and heat-shocked early in their development. If I-SceI endonuclease cuts the chromosome at the site adjacent to the w⁺ reporter, occasional deletions of all or part of the w⁺ gene will occur, and in a white-null background can be identified by the phenotype of eye color mosaicism. The adults that closed exhibited frequent mosaicism indicating loss of w sequences. The results demonstrated that the heat-induced I-SceI can cut a recognition site introduced into the Drosophila genome.

We also carried out quantitative germline assays of I-SceI cutting efficiency by scoring loss of w⁺ in the germline as described herein. The reporter with a cut site adjacent to w⁺ exhibited a low frequency of w⁺ loss, but the construct that was flanked by a tandem duplication of a portion of w showed nearly 90% loss of w⁺, demonstrating that cutting can be quite efficient. The 60-fold increase in the frequency of w⁺ loss with the second tester construct probably does not reflect a real difference in cutting efficiencies, but rather a difference in the preferred route of repair. In the second construct, repair with loss of w⁺could occur efficiently either via a single strand annealing mechanism [Rudin and Haber (1988) Mol. Cell. Biol. 8:3918; Maryon and Carroll (1991) Mol. Cell Biol. 11:3268; Sun, H. et al. (1991) Cell 64:1155) or by homologous recombination between the repeats that flank the cut site. These results indicate that an efficient homologous recombination mechanism exists in germline cells and that the double-strand break can provoke that mechanism.

The coding region of I-SceI was excised from pCMV/SCE1XNLS (a gift from M. Jasin, Sloan-Kettering Institute; 15) as a 900 bp EcoRI-SalI fragment. The EcoRI overhang was blunted by Klenow treatment. This fragment was cloned between the blunted BamHI and the SalI sites of p70ATG→Bam Petersen and Lindquist (1989) Cell Regulat. 1:135]. The resulting plasmid has the I-SceI gene inserted between the Drosophila hsp70 promoter and its 3′ UTR. This 70I-SceI transgene was cloned as a 2.6 kb SalI-NotI fragment into the P element vector pYC1.8 [Fridell and Searles (1991) Nucleic Acid Res. 19:5082]. This gave rise to pP[y⁺70I-SceI. The 18 bp I-SceI cut site (termed I-site here) [Colleaux et al. (1988) supra] was synthesized as two oligonucleotides, ggccgctagggataacagggtaatgtac (SEQ ID NO: 1) and attaccctgttatccctagc (SEQ ID NO:2) that were allowed to anneal to each other and cloned between NotI and KpnI of plasmid pw8 [Claimants, R. et al. (1987) Nucleic Acids Res. 15:3947]. This generated pP[w8,I-site], the tester construct of FIG. 1A. The same synthetic I-site was cloned between the Notl and KpnI sites of pP[X97] [Golic, M. M. et al. (1997) Nucleic Acid Res. 25:3665] to generate pP[X97, I-site]. Each of these constructs was transformed by standard P element-mediated techniques. The FRT-flanked portion of P[X97, I-site] was mobilized to the RS3r4A element on chromosome 2, and to the RS3r-2 element on chromosome 3 by FLP-mediated DNA mobilization (20), generating the tester construct of FIG. 1B in two different locations (Golic M. M., et al., (1997) Nucleic Acid Res. 25:3665).

To test I-SceI cutting, males that carried a transformed copy of 70I-SceI and one of the reporter constructs, with either the reporter-bearing chromosome or its homolog carrying a dominant genetic marker, were heat-shocked for 1 hr at 38° C., at 0-3 days of development. The heat-shocked males that closed were test-crossed individually, and their progeny scored for the eye color. The frequency of w⁺ loss is measured as the fraction of progeny receiving the reporter chromosome that were white-eyed. For the reporter P[w8, I-site], the results of FIG. 1A are the summed results of testing five independent insertions of the reporter that were located on either X, 2, or 3. For the reporter of FIG. 1B, two independent insertions were tested.

### EXAMPLE 2

We designed a transgenic targeting construct (the donor construct) that had an I-SceI cut site placed wit a cloned copy of the Drosophila yellow⁺ (y⁺) body color gene. This gene was also flanked by FRTs (FIG. 2) and the entire assembly inserted with in a P element for transformation. In flies that carry this construct the induction of FLP recombinase and I-SceI endonuclease results in excision of the FRT-flanked DNA to free the donor and cutting of the excised circle to generate a recombinogenic donor.

Two forms of constructs are typically used in gene targeting - “ends-in” constructs or “ends-out” constructs (FIG. 3). Gene targeting in mouse ES cells typically uses ends-out constructs [Mansour, S. L. et al. (1988) Nature 336:348], but the donor element that we built was designed for ends-in targeting. Ends-in targeting can be generally more efficient than ends-out targeting in both yeast and mammalian cells [Hasty, P. et al. (1991) Mol. Cell Biol. 11:4509; Hastings, P. J. et al. (1993) Genetics 135:973; Hasty, P. et al. (1994) Mol. Cell. Biol. 14:8385; Leung, W.-Y et al. (1997) Proc. Natl. Acad. Sci. USA 94:6851]. An ends-in donor construct was chosen to increase the frequency of recovering the desired targeted recombinants. The donor construct shown in FIG. 2 was designed to target the y gene which is located at cytological locus 1B, near the tip of the X chromosome. The expected fate of an ends-in recombinogenic donor molecule was integration at the locus of homology, producing a tandem duplication of the targeted gene as indicated in FIG. 3 [Rothstein, R. (1991) Methods in Enzymol. 194:281]. The targeted locus was the y¹ mutant allele which has a point mutation in the first codon [Geyer, P. K. et al. (1990) EMBO J. 9:2247]. Because the I-SceI cut site in the donor is located to the right of this mutation the result of homologous recombination will be that the right-hand copy of y in such a tandem duplication is y⁺ and the recessive y mutant phenotype will be masked. The result of gene targeting using the described constructs is therefore rescue (recovery of wild-type phenotype) of the y¹ mutation.

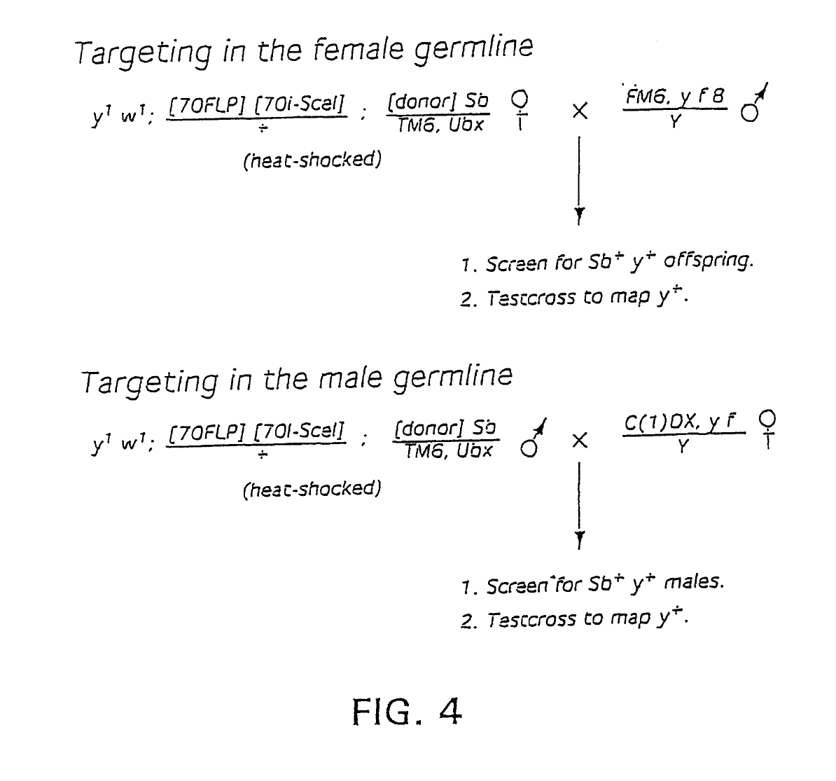

We screened for targeted rescue of y¹ in carrier host flies that carried a heat-inducible FLP gene (70FLP), 70I-SceI, and the donor construct of FIG. 2 (Example 2). We heat shocked those flies early in their development, and then test-crossed and screened for progeny that were y⁺ but did not carry the chromosome on which the donor construct was originally located (FIG. 4). Fifty-six independent y⁺ rescue events were recovered and 55/56 mapped to the X chromosome the locus of the y¹ target (Table 1). Molecular analysis using PCR revealed that in the majority of cases P2t sequences were still present in close proximity to y sequences. Therefore the β2t sequence served as a molecular marker for cytological determination of the site of y⁺ integration. (The β2t and β3t genes shown in FIG. 2 are part of a selection scheme that was not implemented in these crosses.) The β2t gene was used as a probe for in situ hybridization to polytene chromosomes. Five independently recovered y⁺ lines were examined: in all five, β2t sequences

TABLE 1
Independent yellow Rescue Events

| Class | Targeted | Non-targeted |
|---|---|---|
| I | 19 | 0 |
| II | 19 | 0 |
| III | 13 | 0 |
| IV | 4 | 1 |
| Total | 55 | 1 |

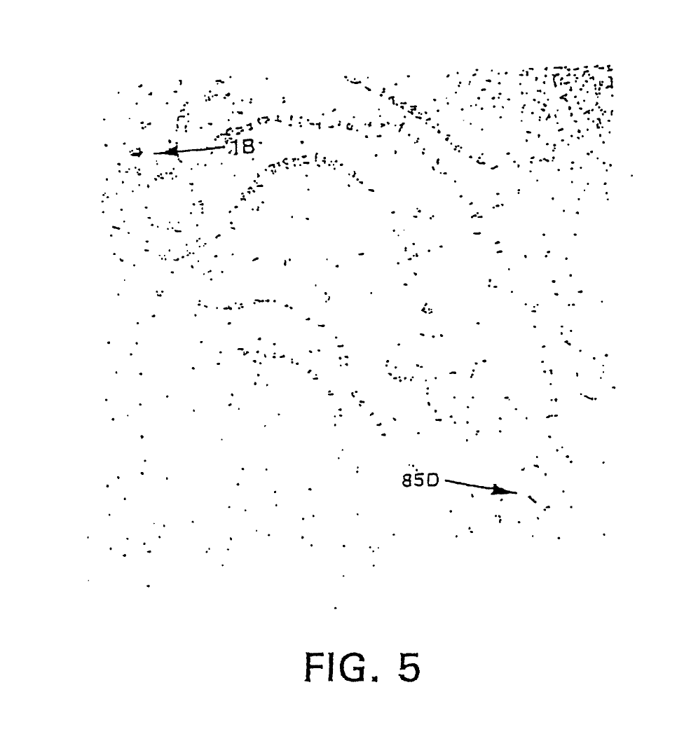

were found at cytological locus 1B in addition to the normal location of the β2t gene at 85D on the right arm of chromosome 3 (FIG. 5), confirming that targeted integration of the donor construct had occurred in the y region.

The y rescue events obtained in the foregoing example occurred far more efficiently in the female germline than in the male germline. Fifty-three independent y⁺ progeny (80 total) were recovered from 224 female test vials for an overall efficiency of approximately one event per 4 vials screened. Each vial produced 100-150 progeny, so the absolute rate was approximately one independent y⁺ offspring for every 500 gametes. Only three events were recovered from 201 male test vials yielding a 16-fold lower efficiency. Because, in Drosophila, a meiotic recombination occurs in females but not in males, these results raise the question of whether efficient gene targeting relies on the machinery of meiotic recombination. In other words, does targeted recombination occur in female meiotic cells? Although our experiments were not specifically designed to address this question, some evidence on this point can be adduced by considering whether the targeting events occur independently or in clusters. Meiotic events are expected to be independent, and exhibit a Poisson distribution. Events that occur in mitotic cells of the germline can be replicated as cells pass through S phase and may produce multiple y⁺ progeny from a single event, leading to clustering of the recovered y⁺ events. The female germline data differed significantly from a Poisson distribution (P<0.001), exhibiting many more clusters than predicted, suggesting that the targeting events occurred pre-meiotically. The non-independent clusters that arose must have occurred many mitosis prior to meiosis, because the last four mitotic divisions in females produce a cohort of cells from which arises a single gamete.

Molecular Analysis.

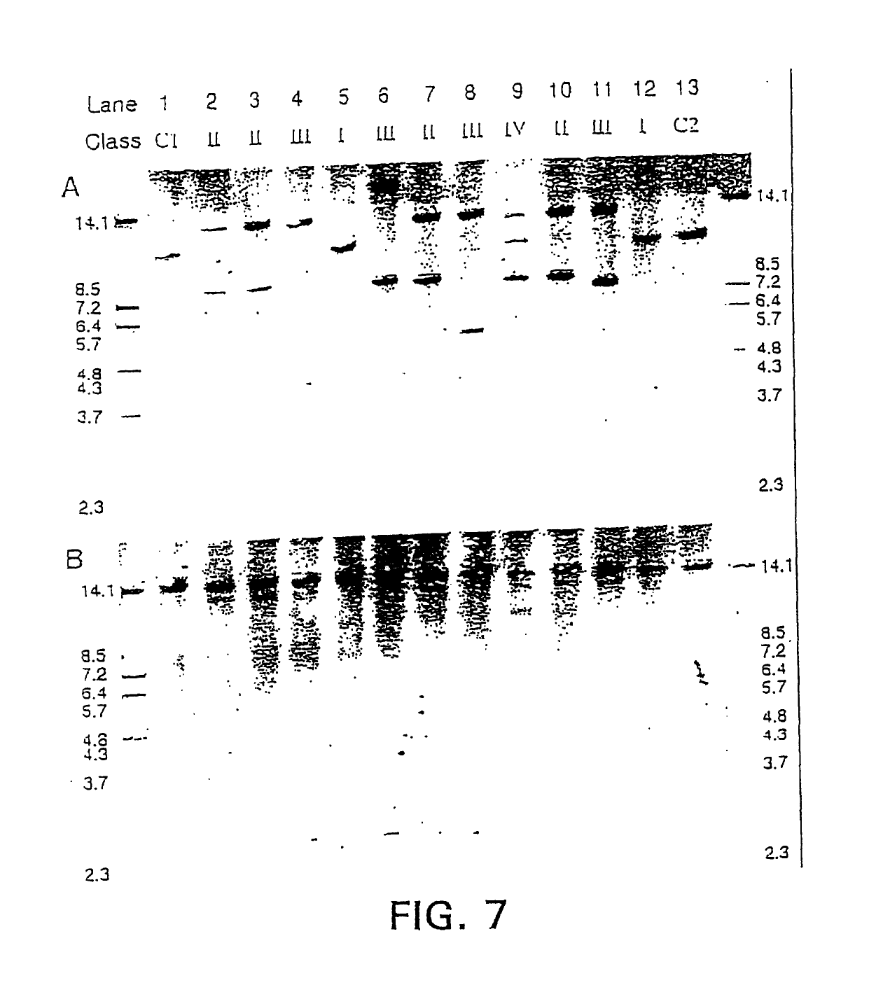

All 56 independent y⁺ lines were analyzed in more detail by Southern blotting. The results showed that the 55 X-linked events were the result of targeted recombination at the y locus. We recovered four classes of targeted events that rescued the y¹ mutation (FIG. 6). The first class consists of simple allelic substitution events that Southern blotting cannot distinguish from the original y¹ allele (FIG. 7). These may have been produced by simple double crossovers between the donor and y¹ (as diagramed in FIG. 6) or by gene conversion.

The second and equally numerous class is composed of tandem duplications of y, with the β2t gene located between the two copies. These almost certainly arose by integrative recombination between the chromosomal y¹ allele and the cut donor as shown in FIG. 6. (Molecular data are shown in FIG. 7.) When the donor element was constructed, the I-SceI cut site was cloned into the SphI site within the intron of y, destroying the SphI site in the process. Sixteen of the 19 Class II alleles had regenerated the SphI sites in both copies of y, demonstrating that the I-SceI recognition site can be readily removed during the recombination reaction, and the site converted to the sequence of the targeted locus.

The high frequency of Class II tandem duplications suggests another route by which the Class I events may have been produced. Recombination between directly repeated y genes at a site to the left of the mutation in y¹ would reduce the duplicate genes to a single copy of y⁺. In previous experiments, small tandem duplications that we have generated are very stable (for example the P element of FIG. 1B; also references Golic and Lindquist (1989) supra, and Golic and Golic (1996) Genetics 144:1693]. If Class I events do occur by this route it is likely that it immediately follows the integration event when nicks or breaks are still present. As FIG. 1 shows, tandem duplications are readily lost when a ds break is introduced between the duplicate copies.

The third class consists of tandem duplications of y with insertions or deletions of material in one of the two copies (FIG. 6). These alterations occur about the location at which the I-SceI cut site was placed. Although we have not identified the additional DNA that is present in the insertion alleles, the stronger hybridization signal exhibited by the upper band in lane 6 (FIG. 7) suggests that in at least some cases it is from the y gene. The Class m events may arise by imprecise initiation or resolution of the recombination reaction.

The fourth and least frequent class consists of y¹ rescue events resulting from the integration of two additional copies of y (FIG. 6). Five such events were recovered: four were targeted to yellow and produced a triplication of the gene, and one occurred on chromosome 3. Although our experiments used flies with only a single donor transgene, when a cell is in G2 two copies of the donor will be present. The two copies on sister chromatids might dimerize through FLP-mediated unequal sister chromatid exchange [Golic and Lindquist (1989) supra], or by end-joining of two independently excised and cut donor molecules. Integration of such a dimer could produce the observed results. Although all three bands detected with a y probe should hybridize with equal efficiency, the class IV event shown in FIG. 7 (lane 9) shows a stronger hybridization signal on the 8.0 kb band than on the 10.5 and 12.5 bands. This particular event may carry yet a fourth copy of y. The remaining four class IV recombinants appear to be the simpler events diagramed in FIG. 6.

In these mutation-rescue experiments, the donor DNA was cut in the middle of the wild-type rescuing allele. To generate a chromosomal y⁺ gene, recombination that is stimulated by the cut must almost inevitably occur with the y¹ allele. If a single copy of the donor were to integrate elsewhere it seems highly unlikely that a functional copy of y⁺ would be produced. Thus, our screen practically demands that only integration events targeted to y would be detected, and Class I, II, and m events give no information on the relative frequencies of targeted events versus random insertions. However, the recovery of Class IV events allows us to examine this issue because the middle copy of y⁺ should be functional even when the donor molecule integrates, not by recombination with y, but at some other site. Class IV events should be recoverable whether targeted to y or not. We recovered five Class IV events and four of the five had integrated at the normal location of y on the X chromosome. Therefore, even in cases where it was possible to detect integration at sites other than y, the majority of recombinants were targeted to y. The single non-targeted Class IV integrant was located on chromosome 3 but did not appear (by Southern blotting) to be targeted to the β2t gene.

The results demonstrate that randomly inserted transgenes can be converted to targeted insertions through the use of a site-specific recombinase and unique site-specific endonuclease. The method was quite efficient, allowing targeting events to be identified simply by a genetic linkage screen, and produced an average of one targeted recombinant for every 4-5 vials examined (in females). Our screen detected events that used a donor DNA to convert a mutant allele to wild type. The same basic method, modified by the choice of donor construct and selection method can be used to generate any desired modification of a target gene even if the target gene is known only by the sequence. Essentially any gene of the Drosophila genome can be targeted, using data from the published Drosophila genome sequence [http://www.fruitfly.org/.l It will be apparent to those skilled in the art that the technique developed is readily adaptable to targeting any gene or DNA segment whose sequence is known. Many of the techniques that have been developed for disrupting genes in yeast are adaptable for analogous application in Drosophila [Rothstein (1991) supra].

### EXAMPLE 3

The data of Examples 1 and 2 do not rule out the possibility that the targeted gene modification observed relied on a type of DNA repair termed Break-induced Replication (BIR). Hypothetically, a single one-ended homologous exchange may have occurred, leaving the recombinant chromosome with a truncated terminus. In order to be recovered as a viable product this chromosome with a modified target locus would be repaired by BIR, wherein the broken terminus invades the homolog prompting unscheduled replication to the end of the chromosome [see, e.g. Engels, W. R. (2000) Science 289:1973]. Since the yellow gene that we targeted lies approximately 110 kb from the X chromosome telomere, it is not unreasonable to imagine that a chromosome break at this location could be repaired by replication to the end of the chromosome. Additionally, targeting was much more efficient in the female germline (with two X chromosomes) than the male germline (with one X), and the BIR model, wherein repair of a one-ended recombination event relies on replication templated from a homolog, provides an explanation for this difference. Finally, the classes of targeting events that we recovered could be explained both by homologous recombination, or by a combination of homologous exchange and BIR. The significant implication of the foregoing explanation is that, if targeting must involve BIR, then it is likely that only genes situated near telomeres can be successfully targeted because of the requirement for continuous replication to the end of the chromosome. Thus, it is useful to know whether the technique of the invention can be applied broadly, or whether it will be limited to genes near telomeres.

Straightforward homologous recombination is a more parsimonious explanation for our data. In considering the hypothesis that the gene targeting described in Examples 1 and 2 relies on BIR, and secondarily on the presence of a homolog, one cannot overlook the fact that genuine targeting events, although small in number, were recovered from males. These males of course have but a single X chromosome. Furthermore, if a one-ended homologous recombination event can occur there is no obvious reason why two-ended events should not occur. The following experiment was performed to test the foregoing hypothesis. Data from the experiment, described herein, demonstrate that we have generated a targeted knockout of a gene that is very far removed from telomeres. Consequently, the hypothesis just described does not account for the observed results and the method of the invention has been shown to be broadly applicable for any target gene.

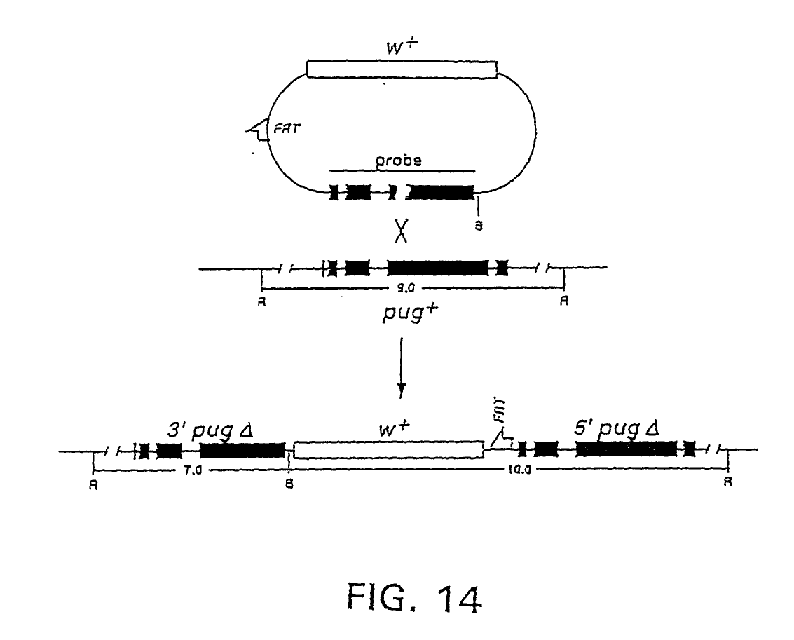

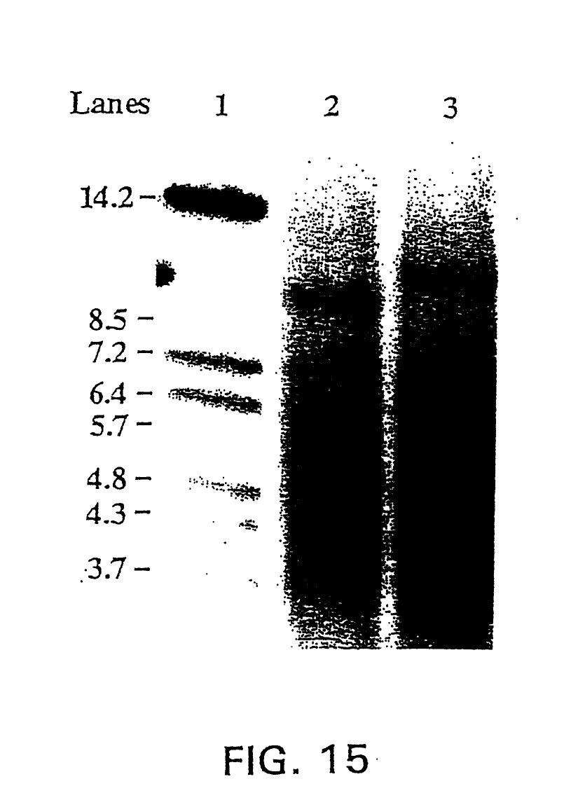

The pugilist (pug) gene encodes a homolog of the trifunctional form of the enzyme methylene tetrahydrofolate dehydrogenase, and animals carrying mutations in this gene show eye color defects [Rong et al. (1998) Genetics 150:1551]. The gene is located at 86C on the right arm of chromosome 3 approximately 20 Mbp from the nearest telomere. A 2.5 kb fragment of the gene was engineered lacking the first, and part of the fourth and fifth exons, by inserting a recognition site for I-SceI endonuclease at an ApaI site in exon 4, and placed it into the P element vector P[>w^{hs}>] [Golic et al. (1989) Cell 59:499]. In this vector, the engineered pug fragment and w^{hs} are flanked by direct repeats of the FLP Recombination Target (FRT). Transformants were generated and crossed to produce flies that carry 70FLP, 70I-SceI and the pug donor construct. We heat-shocked these flies as described herein [see also Rong et al. (2000) Science 288:2013 incorporated herein by reference in its entirety] and carried out a segregation screen to look for mobilization of the w^{hs} marker gene to a different chromosome. From 455 female vials we recovered 3 independent cases of w^{hs} mobilization. Two of the events were instances of pug knockout produced by targeted recombination between the donor DNA and the resident pug⁺ gene (FIG. 14). The pug allele at the left (3′ pug Δ) carries a deletion which includes part of exon 4, exon 5 and 3′ UTR of pug. The pug allele at the right (5′ pug Δ) lacks the promoter and exon 1 of pug. Three criteria support this conclusion: Southern blotting (FIG. 15) showed bands of the sizes expected for a Class II targeting event [Rong et al. (2000) supra]; in situ hybridization showed that the w^{hs} gene was now located at 86C; and the targeted alleles exhibited the pug null phenotype. The remaining event was an integration at a site other than pug and was not examined further.

The results of the pug targeting experiment do not rule out the possibility that some of the targeting events we previously reported at yellow did arise by homologous recombination and BIR. The explanation for the difference in targeting efficiency between pug and yellow is most likely due to the different amounts of donor:target homology in the two experiments—8 kb in the yellow experiments vs. 2.5 kb in the pug targeting experiments reported here.

The results of the pug targeting experiment also show that non-targeted insertions, although they do occur, are not so frequent as to be a significant nuisance. Here, the targeted recombinants outnumbered the non-targeted recombinants by 2:1. If targeting efficiency is improved, for example by increasing donor: target homology, then non-targeted events would constitute an even smaller portion of events detected by the segregation screen. Tending to confirm this supposition, in the yellow targeting experiments a majority of the informative Class IV events were a result of targeted recombination Rong et al. (2000) supra].

Most importantly, the results presented here demonstrate that non-telomeric genes can be targeted and modified by homologous recombination, and this can be done solely by following the inheritance of an arbitrary marker gene.

### EXAMPLE 4

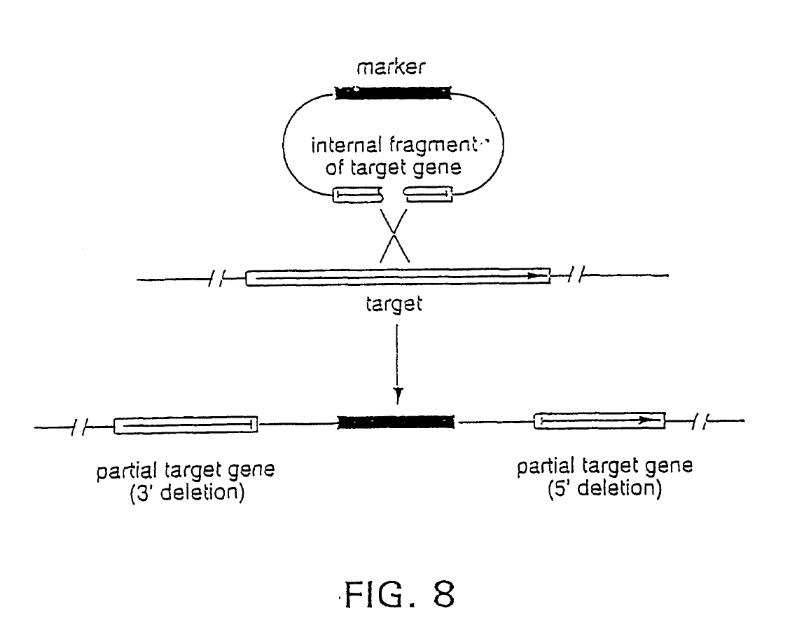

Another embodiment of the method for targeted mutagenesis is diagramed in FIG. 8. A fragment of the gene to be mutated has an I-SceI or other unique endonuclease cut site placed within it. This donor DNA and a marker gene is placed between FRTs and then into a transposon vector for transformation. After induction of FLP and I-SceI in females, targeting events can be detected by altered linkage of the marker gene, and verified by genetic or molecular techniques. As we have shown in our screen the targeted events outnumbered non-targeted events. Thus, it will be relatively easy to recover the desired recombinants. In the example of FIG. 8, a Class II integration event produces two truncated mutant alleles.

Many of the targeted events that we recovered in the first described embodiment were not produced by precise recombination. The Class III events had alterations in the targeted locus that would not be predicted by homologous exchange. Some of the Class II events may also have very small alterations that were not detectable by Southern blotting. It is also likely that there were many additional Class m targeted events that were not recovered in our screen because they carried deletions that destroyed the y⁺ locus. So, although gene targeting often resulted from precise recombination there are also many imprecise and potentially mutagenic events. It follows that is it not necessary that the donor construct carry a mutant form of the target locus (such as the truncated gene of FIG. 8). Mutant alleles can be produced at a reasonable rate simply by imprecise targeting events. Such a result has precedence in the examination of stably transformed Drosophila cell lines. Cherbas and Cherbas [(1997) Genetics 145:349] observed that in many cases, DNA transfected into cell lines had integrated near the chromosomal locus with homology to that DNA, and that rearrangements were often produced that in some cases generated mutations of the chromosomal locus. They termed the phenomenon parahomologous targeting and it may be closely related to the processes that are responsible for the Class m events that we recovered.

As previously described, an I-CreI cut site may also be introduced, which allow the reduction of class III alleles to a single copy mutant allele.

The invention makes it possible to introduce point mutations and a variety of other changes. Moreover, the not infrequent occurrence of Class I events indicates that it is feasible to produce allelic substitutions at other loci. Finally, the frequent replacement of the I-SceI cut site sequences at the termini of the donor with the wild-type genomic sequence indicates that it is feasible to carry out targeting with an I-SceI cut site placed within a gene's coding sequence, and yet not necessarily destroy that portion of the gene.

### EXAMPLE 5

The procedures of Examples 1 and 2 were modified in two ways to adapt the invention to plants. First, we used the Cre/Lox recombination system in place of the PLP/FRT recombination system. The Cre/Lox system was utilized since prior studies in the laboratory made the starting constructs immediately available. The Cre/Lox system has been demonstrated to work well in plants [Sieburth, Drews and Meyerowitz (1998) Development 125:4303]. The FLP-PRT system, however, can work equally well according to the literature. Second, we utilized plant specific promoters to drive expression of the Cre and I-SceI genes (discussed below). The gene targeting is described for Arabidopsis because its short generation time, ease of transformation, and small genome make it a convenient model for gene targeting in plants.

In adapting the method of the invention to plants (as to any organism) aspects of the organisms biology should be taken into account. Specifically, plants have a different pattern of development from animals which affects the developmental stage when homologous recombination is most likely to occur. The most important difference is that plants lack a “germ line” in the sense of an animal germ line. In animals, a specific set of cells (the germ line cells) is set aside early in development to become the germ cells. In plants, no such event occurs. Plants develop via meristem growth. The shoot apical meristem at the tip of the plant contains a group of rapidly-dividing cells that give rise to the entire above-ground portion of the plant (i.e., the entire shoot) including the flowers. At a specific time of development, the shoot apical meristem gives rise to floral primordia. Floral primordia develop into flowers containing four organ types: sepals, petals, stamens, and carpels. Inside the stamens and carpels are produced the microspore mother cells and megaspore mother cells, respectively. The mother cells undergo meiosis to produce haploid microspores and megaspores, which develop into the haploid male and female gametophytes that contain the sperm and egg cells, respectively.

Thus, for an homologous recombination event to be transmitted to the following generation, it is preferred to express the Cre Recombinase and I-SceI enzymes in one of the following patterns: (1) the zygote, (2) the embryo cells that give rise to the shoot apical meristem, (3) the portion of the shoot apical meristem that gives rise to the germ cells (the L2 layer in most species), (4) the cells of a developing flower that give rise to the mother cells, (5) the mother cells, (6) the developing gametophytes, (7) the egg and/or sperm, or (8) cultured cells.

A convenient place to induce homologous recombination is in the mother cells that give rise to the germ cells. First, homologous recombination occurs at elevated frequency in cells undergoing meiosis because this is the time when meiotic homologous recombination normally occurs. Therefore, the enzymes needed to carry out the process are clearly present and functional in these cells. Second, because each mother cell gives rise to a different gamete, each mother cell represents an independent “attempt” at homologous recombination. Finally, each plant produces thousands of mother cells; thus, thousands of homologous recombination “attempts” occur in each plant.

By contrast, gene targeting by homologous recombination in the shoot apical meristem is likely to occur at a lower frequency, but may still be used in the invention. The shoot apical meristem cells divide rapidly and are less likely to contain the enzymes required to undergo homologous recombination.

Two promoters were used to drive expression of the Cre Recombinase and I-SceI genes in Arabidopsis. The first is the promoter from the Arabidopsis ATDMC1 gene [Klimyuk and Jones (1997) Plant Journal 11:1-14]. This promoter directs expression to the pollen mother cells and megaspore mother cells. As described above, directing expression of the Cre and I-SceI genes to the mother cells has several advantages. The second promoter used is the promoter from the Arabidopsis HSP 18.2 heat shock gene [Takahashi and Komeda (1989) Mol. Gen. Genet. 219:365-372]. This promoter provides inducible expression in Arabidopsis, which is convenient for testing various developmental stages for effectiveness of obtaining homologous recombination. This promoter has been used to drive expression of the Cre Recombinase gene in Arabidopsis [Sieburth et al. (1998) Development 125:4303-4312]. Four enzyme constructs were made as summarized in the table below:

| Construct Name | Promoter | Gene |
|---|---|---|
| DMC1::Cre | AtDMC1 | Cre |
| DMC1::ISceI | AtDMC1 | I-SceI |
| HS::Cre | HSP 18.2 | Cre |
| HS::ISceI | HSP 18.2 | I-SceI |

HS = heat shock promoter
DMC1 = AtDMC1 promoter

In addition to the above, other promoters can be utilized, for example, other useful promoters include LEC1 (lotan et al. (1998) Cell 93, 1195-1205), which confers expression in the zygote and early embryo; the CaMV 35S promoter, which confers somewhat constitutive expression and will induce homologous recombination in the cells that give rise to the shoot apical meristem, and the SHOOT MERISTEMLESS (Long et al., (1996) Nature 401, 769-777) and CLAVATA3 (Fletcher et al. (1999) Science 283, 1911) promoters that will drive expression in the L2 layer of the shoot apical meristem. A preferred promoter is one that can drive expression in the L2 layer, which contains the shoot apical meristem cells that give rise to germ cells. Candidates include STM, CLV1, CLV2, CLV3.

The present example employs gene targeting to convert a mutant allele into a wild-type allele. This approach obviates the need to include a complex selection strategy. The targeting is demonstrated with two genes that have well-defined and easily-scored mutant phenotypes, and that are transformable at high frequency. The genes are the Arabidopsis CRABS CLAW1 (CRC1) gene [Bowman and Smyth (1999) Development 126:2387-2396] and the Arabidopsis CLAVATA1 (CLV1) gene [Clark et al. (1997) Cell 89:575-585]. Donor constructs include a wild-type copy of the gene with an I-SceI site in an exon flanked by loxP sequences. We have made two donor constructs as summarized in the table below:

| Construct Name | Gene |
|---|---|
| CRC1-D | CRC1 |
| CLV1-D | CLV1 |

The general structure of the donor construct is as follows:

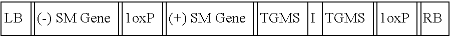

| | |
|---|---|
| LB = | left border of T-DNA |
| (−)SM Gene = | negative selectable marker gene (optional) |
| (+)SM Gene = | positive selectable marker gene (optional) |
| TGMS = | target gene modifying sequence |
| I = | I-SceI site within the target gene modifying sequence |
| RB = | right border |

While this example describes a method of converting a mutant allele to a wild-type allele, other types of conversions are within the scope of the invention. One such conversion involves the converting a wild-type allele to mutant allele, which can in certain instances involve the use of selection schemes to recover organisms in which the targeting has occurred.

Such selection schemes can advantageously employ selectable markers. The negative selectable marker gene used herein is the E. coli codA (cytosine deaminase) gene [Mullen et al. (1982) PNAS 89:33-37; Mullen and Blaese (1994) U.S. Pat. No. 4,975,278; Stougaard (1993) Plant Journal 3:755-761; Serino and Maliga (1997) Plant Journal 12:697-701]. A variety of other negative selectable marker genes are available including the Agrobacterium tms2 gene [Depicker et al. (1998) Plant Cell Rep. 7:63-66 the nitrate reductase gene [Nussaume et al. (1991) Plant Journal 1:267-274], and the alcohol dehydrogenase gene. The positive selectable marker gene used herein is the neomycin phosphotransferase gene, which confers resistance to kanamycin [Fraley et al. (1998) PNAS 80:4803-4807]. Many other positive selectable marker genes are available and known to those of ordinary skill in the art.

Various modifications to the foregoing procedure can be introduced to simplify and streamline the process. The number of generations to obtain a homozygous mutant can be reduced by instituting two changes. The first is to introduce the donor constructs into a carrier host, a plant strain that already has been transformed with the enzyme constructs. This change will decrease the number of generations to three. The second change is to utilize promoters to drive expression of the Cre Recombinase and I-SceI genes very early during embryo development, ideally in the egg cell of zygote. The combination of changes reduces the number of generations to two.

The time required to make donor constructs can be reduced by constructing a cloning vector to simplify cloning the target modifying sequence. The modifying sequence cloning site (CS) contains an I-SceI site flanked by two sites for target modifying sequence cloning (Tm-L, left TM cloning site; TM-R, right TM cloning site). It also has a multiple cloning site (MCS) containing several unique restriction sites.

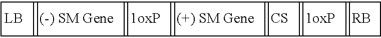

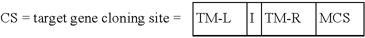

In addition to the above, it is possible to induce homologous recombination at the moment of T-DNA integration. With in panta transformation, it is thought that it is the egg cell that becomes transformed. The donor construct is introduced into a plant stain expressing the Cre recombinase and I-SceI endonuclease genes in the egg cell. Doing so confers the advantages of saving one generation of time to obtain a plant homozygous for the gene modification.

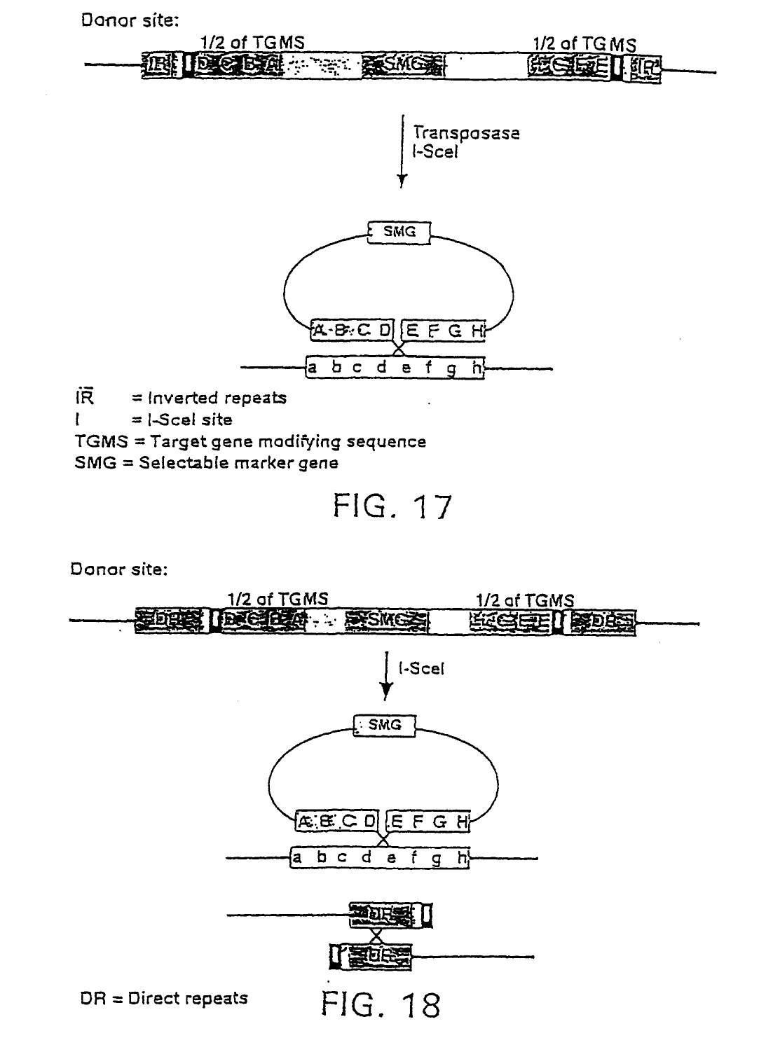

It is also possible to use a transposon to excise the target gene. This obviates the need for using the Cre-lox or Flp-FRT system to do so. The transposase and I-SceI endonuclease are expressed at the same time. The transposase excises the transposon and then I-SceI endonuclease cuts at the I-SceI sites. These cuts create the same situation that is obtainable with the Cre-lox or Flp-FRT system (see FIG. 17). Again, it can be advantageous to express transposase/I-SceI in the mother cells, just before or during meiosis.

Introducing the Constructs into the Arabidopsis Genome:

We have introduced all constructs into the Arabidopsis genome using Agrobacterium-mediated transformation. Each construct was assembled in an E. coli plasmid vector (pBluescript or other) and then ligated into the pCGN1547 Binary Ti-plasmid transformation vector McBride and Summerfelt (1990) Plant Molecular Biology 14:269-276]. The pCGN1547 clone was first introduced into E. coli and then into Agrobacterium strain ASE. Agrobacterium strains containing the various constructs were used to infect mutant (clv mutants or crcl mutants) Arabidopsis plants using in planta transformation [Chang et al. (1994) Plant Journal 5:551-558; Bechtold et al. (1993) C.R. Acad. Sci. Paris Life Sci. 316:1194-1199; Clough and Bent (1998) Plant Journal 16:735-743; Katavic et al. (1994) Mol. Gen. Genet. 245:363-370]. In this procedure, Arabidopsis plants are dipped in an Agrobacterium solution and the plant reproductive tissues become invaded by the bacteria. Optinal heat shock conditions may vary from strain to strain. Testing and determination of heat shock conditions can be performed by one of ordinary skill in the art. It is thought that the egg cell becomes transformed [Ye et al. (1999) Plant Journal 19:249-257; Bechtold et al. (2000) Genetics 155:1875-1997]. Transformed strains were selected for kanamycin resistance. Using this procedure, we have generated six Arabidopsis strains:

| Strain Name | Genetic Background | Introduced Construct |
|---|---|---|
| clv1-HSE | clv1 | HS::Cre and HS::ISceI |
| crc1-JSE | crc1 | HS::Cre and HS::ISceI |
| clv1-DCME | clv1 | DMC1::Cre and DMC1::ISceI |
| crc1-DCME | crc1 | DMC1::Cre and DMC1::ISceI |
| clv1-D | clv1 | CLV1 Donor Construct |
| crc1-D | crc1 | CRC1 Donor Construct |

HS = heat shock promoter
DMC1 = AtDMC1 promoter

These strains were grown and crosses were carried out to bring together the enzyme constructs and donor constructs. Specifically, the following crosses were carried out;

(1) Strain clv1-HSE×Strain clv1-D;

(2) Strain clv1-DCME×Strain clv1-D;

(3) Strain crc1-HSE×Strain crc1-D; and

(4) Strain crc1-DCME×Strain crc1-D.

Inducing Recombinase and Endonuclease Enzyme Expression:

In the strains harboring the heat shock promoter-enzyme constructs, induction is carried out by immersion in warm water as described by Sieburth et al. (1998) Development 125:430-4313. Heat induction is carried out at a variety of developmental stages including developing embryos (to induce in the cells that give rise to the shoot apical meristem), the tips of floral stems (to induce in the cells of the shoot apical meristem), developing flowers (to induce in the cells that give rise to the mother cells), flowers undergoing meiosis (to induce in the mother cells), and mature flowers (to induce in the germ cells).

In the strains harboring the DMC1 promoter-enzyme constructs, expression is not externally induced. As described above, the developmentally-regulated promoter induces expression of the enzymes at a time just before meiosis.

Identifying Plants in which HR has Occurred:

Plants that have been induced are allowed to undergo self-pollination and progeny seed are collected. The progeny seed are grown and scored for the mutant phenotype. Plants in which targeting has occurred are wild-type. Genotype is verified using PCR.

### EXAMPLE 6

Ends-out targeting in some instances may be preferable to ends-in targeting. It can simplify the construction of the donor element and provide a faster and simpler route to the generation of deletions with precise endpoints. These deletions can also carry a dominant marker gene which can simplify their use in subsequent crosses.

Targeting Yellow by Ends-out Methods

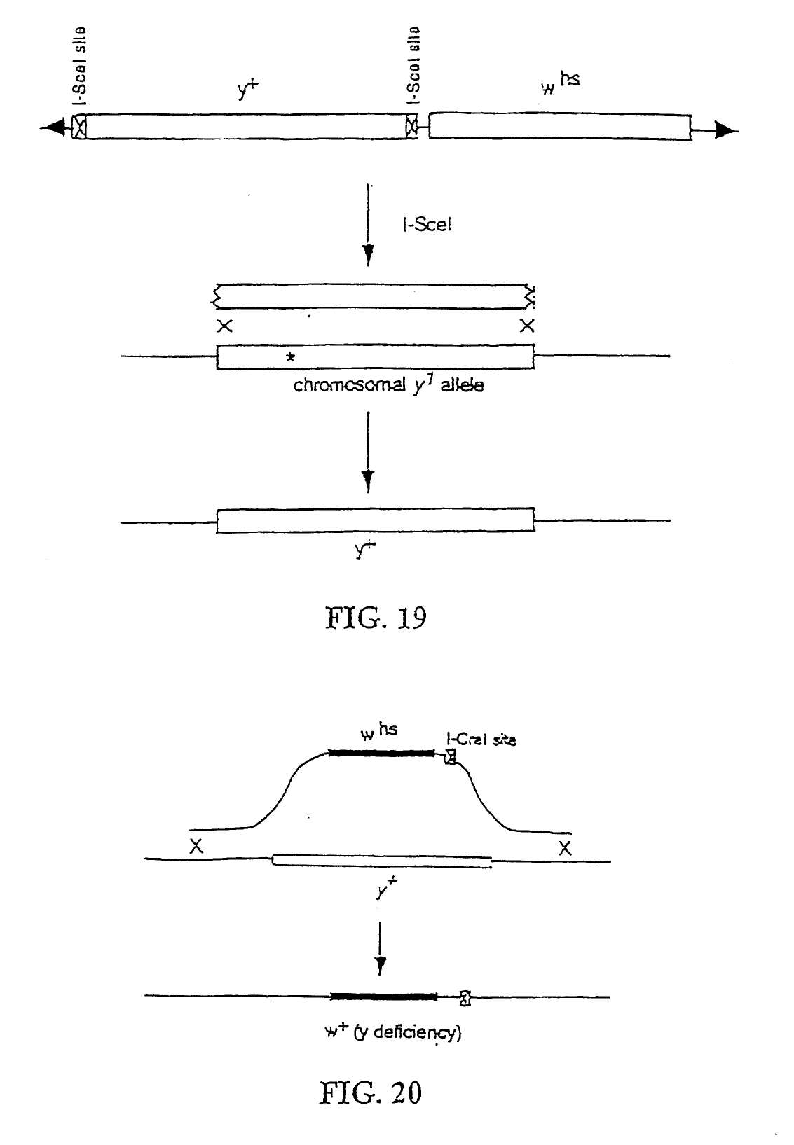

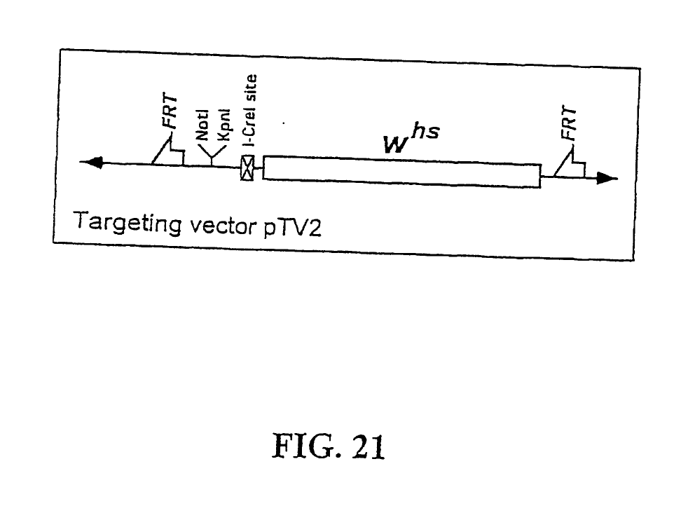

The efficiency of ends-out targeting can be measured with yellow. The donor element is constructed by placing two I-SceI cut sites into the polylinker of the P vector pw8 and then cloning the 8 kb y⁺ fragment between those sites. After transformation and crossing to 70I-SceI flies, I-SceI expression in the offspring is induced by heat shock. A linear DNA fragment comprising the y⁺ gene is freed by double-cutting with I-SceI. See FIG. 19. The heat-shocked flies are then mated and screened for progeny that are y⁺, but not w⁺. These can arise from targeted recombinants at yellow or non-targeted insertions elsewhere in the genome. It is also possible to lose w⁺ function from within the P element by single cutting near w^{hs} and loss of part of the w^{hs} gene to exonucleolytic digestion. Therefore, it is required that the y⁺ w - events map to a different chromosome to be demonstrative examples of y⁺ mobilization. The structures of any y⁺ genes that map to the X chromosome (potential targeting events) are characterized by Southern blotting.

This event relies on two I-SceI cuts rather than a single cut. Since the efficiency of single-cutting is approximately 90% for a single I-SceI site following heat-shock induction of 70I-SceI, it is estimated that ˜80% of the cells experience a double cut. An independent estimate of the efficiency of double-cutting can be provided by scoring the frequency of complete yellow gene loss that arises from the double cut with this ends-out construct. The frequency of double-cutting can be increased by using two or more copies of 70I-SceI.

The ends-in targeting scheme of Examples 1 and 2 allows for repair of an I-SceI cut by FLP-mediated recombination, either before (in which case the cut occurs on an extrachromosomal molecule) or after scission. The described ends-out construct provides no such built-in mechanism to restore the cut chromosome, so that cell death might occur in some instances. Cell death is unlikely for the following reasons: first, when an unrepairable chromosome break is generated by breakage of a dicentric chromosome (because only a single broken end is present), the result in the soma is cell death [Ahmad and Golic (1999) Genetics 151:1041-51]; second, following I-SceI expression in flies carrying a single cut site, little or no cell death is observed. Thus, the chromosome from which the donor is excised is likely to be repaired.

Alternatively, a new version of the donor in which the I-SceI site-flanked yellow⁺ gene is also flanked by FRTs can be used. This construct can be used for ends-out targeting using I-SceI and FLP expression together. When FLP acts first, it will excise the donor, leaving behind an intact chromosome. The donor can then be cut by I-SceI.

Precise deletion of yellow⁺ can be generated using a replacement strategy. Upstream and downstream regions of yellow are cloned to flank a w^{hs} gene and I-CreI recognition site, and this assembly placed between I-SceI sites.

After transformation, a segregation screen for mobilization of w^{hs} to the X chromosome in a y⁺ w background is performed. A targeted recombination event results in the precise deletion of yellow and insertion of w^{hs} in its place (FIG. 20). Recombinant products can be characterized by Southern blotting.

Serial Substitution

One use of ends-out targeting in yeast is to first insert a marker gene into the target locus and, in a second step, replace that marker with an altered allele of the gene in question, followed by screening (or selecting) for loss of the marker. A similar scheme can be carried out in flies by making use of the I-CreI cut site that was included next to w^{hs}. Cutting at this site can stimulate replacement of the w^{hs} marker with sequences from a donor template by gene conversion.

Replacement of the w^{hs} gene is accomplished at the yellow locus by exchanging it for a modified y⁺ allele. They gene missing part of the intron, including the tarsal enhancer, includes y flanking regions to provide the homology for exchange. The crosses can be carried out with a variety of yellow alleles on the homolog (including deletions or y⁺ alleles) by distinguishing homolog-templated events from those that use the introduced gene as a template. The molecular structures of white loss events that are yellow (possibly resulting form gap enlargement and end-joining or incomplete gene conversion) or yellow⁺ (resulting from templated gene conversion) can be examined.

Banga and Boyd [(1992) Proc. Natl. Acad Sci. USA 89:1735-9] and Gloor et al.[(1996) Mol. Cell. Biol. 16:522-8] have shown that injected DNAs can be used as template for P-gene conversion. Thus, alternatively, co-injection of a helper I-CreI gene or I-CreI mRNA can be used to generate a stable transformation through cutting of the chromosome and stimulation of gene conversion. Since the I-CreI cut site in the ends-out-modified yellow locus is not flanked by large direct repeats, as with an ends-in targeting event, there is not likely to be a strong preference for eliminating w^{hs} by intramolecular recombination, and allele-swapping by gene conversion may constitute a large faction of all events that lose w^{hs}.

The length of a span of DNA that can be deleted by the ends-out targeting can be determined using the hsp70 loci as a diagnostic test. These genes are present in two clusters at 87A and 87C and span 6 kb and 50 kb. Unique sequences to the left and right of each cluster can be used for targeting. Alternatively, autosomal targets can be chosen.

Implementation of positive-negative selection can be used to eliminate non-targeted recombinants, which constitute the majority of events in mouse ES cells, but are a minor fraction of events in drosophila

The standard method for detecting targeting events involve detecting the movement of a marker gene from one chromosome to another.

Elimination of Mapping and Marking Steps as Prerequisite for Targeting.

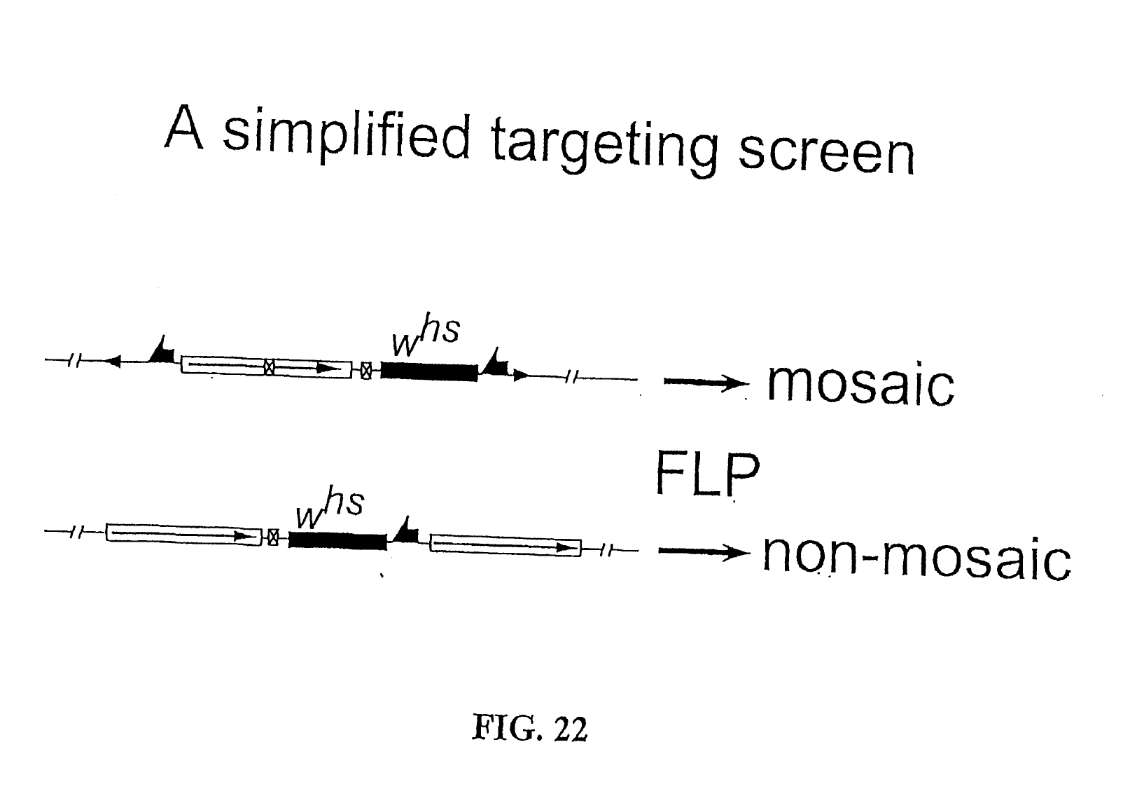

More specifically, the signal for a targeting event is mobilization of the donor from a dominantly-marked chromosome to a different chromosome where the target locus resided and was recognized by segregation of markers in a test-cross. The need for mapping and marking the donor element-bearing chromosome causes a substantial time delay for producing a fly with a modified target gene. By taking advantage of a structural difference between the original donor element insertion and a Class II targeting event, the procedure can be shortened significantly. For example, in a transformed copy of TV2, the targeting construct and the w^{hs} are flanked by FRTs (see FIG. 20 for the structure of the targeting vector). In a class II (or III or IV) targeting event, there is a copy of w^{hs} that is not flanked by FRTs. The mosaicism, or lack thereof, that is produced by FLP can be used as a criterion for distinguishing flies with the original TV2 insertion from flies with a targeting event (see FIG. 22).

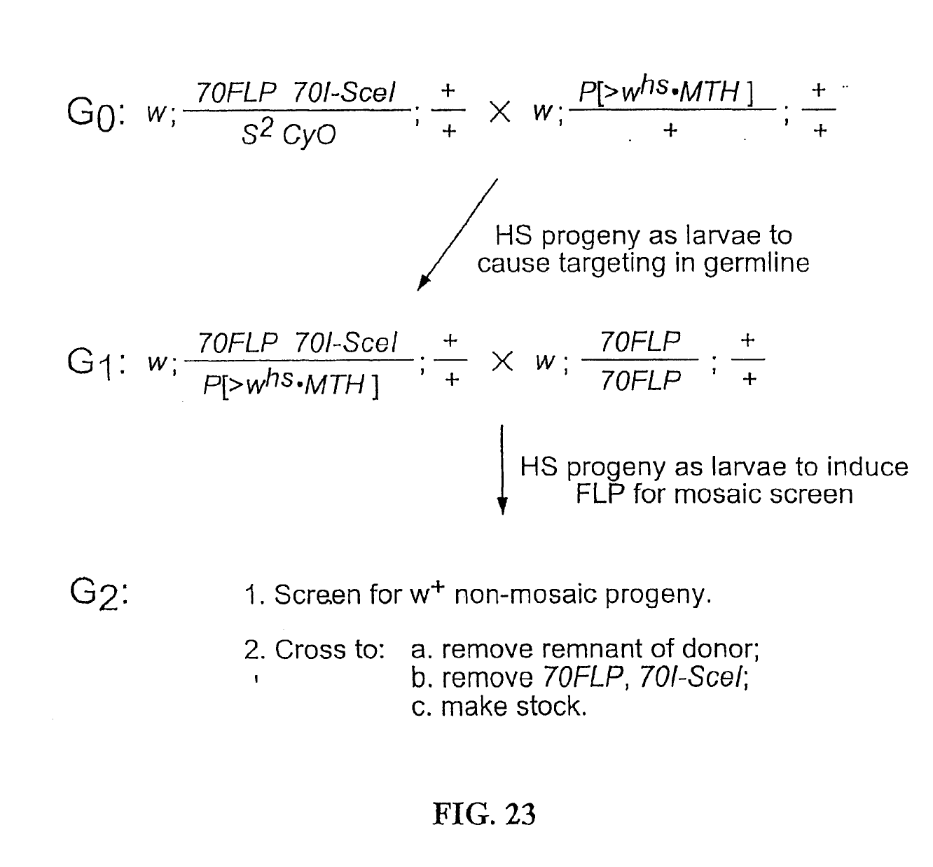

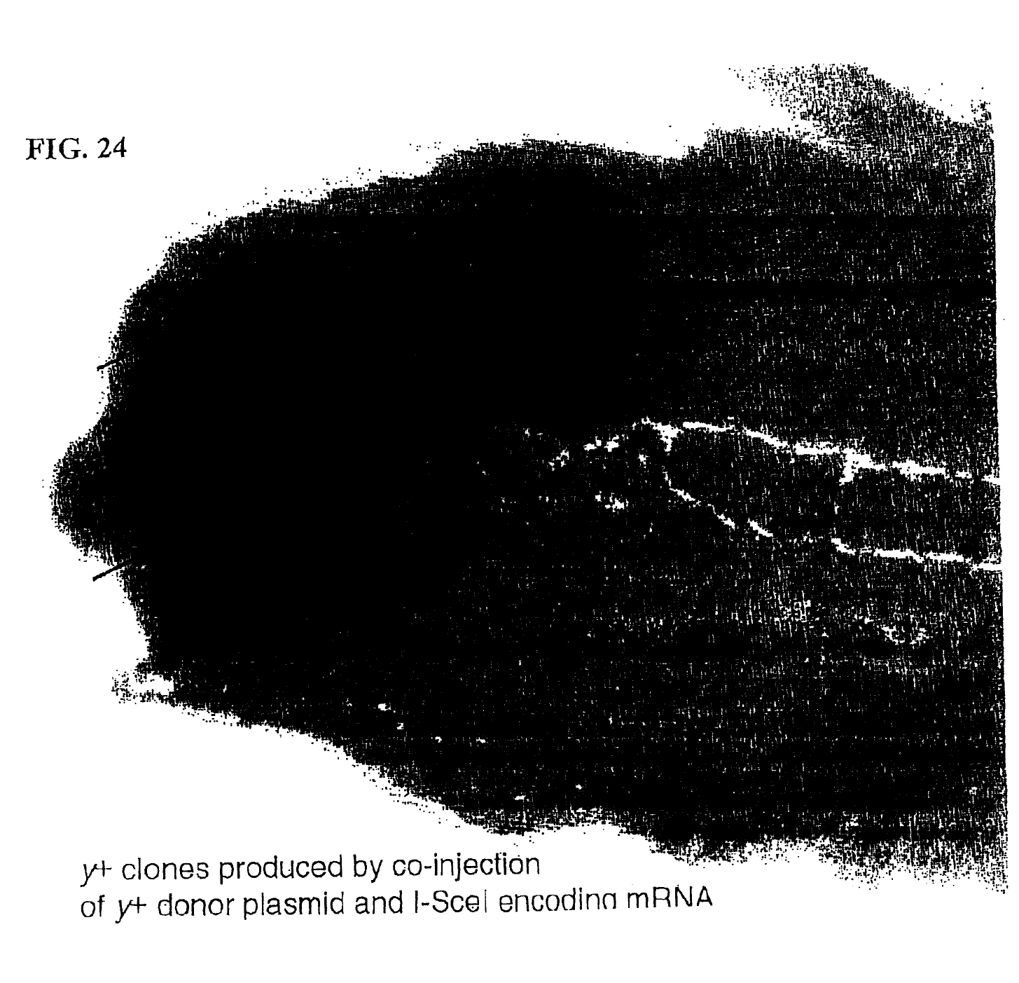

Flies that carry 70FLP, 70I-SceI and the targeting construct are heat-shocked and crossed to flies that are homozygous for an insertion of 70FLP that show a high degree of expression without heat shock (see FIG. 23 for crossing scheme). Most progeny are entirely white-eyed owing to excision and loss of the donor construct carrying a w^{hs} gene. Some progeny with eye pigment can arise from the infrequent failure of excision; these appear as mosaics owing to FLP expressed from the constitutive 70FLP transgene. Targeting events produce progeny with solidly pigmented eyes (as does non-targeted insertion). Targeting is verified by a backcross to the constitutive 70FLP strain; progeny with a lack of mosaicism are characterized by Southern blotting to confirm that they were produced from the expected targeting events.

Due to the efficiency of FLP-mediated excision, the number of false positives can be very low. This screen requires the same number of generations as the original segregation screen, but the step requiring mapping, marldng, and making of stock transformants is completely eliminated as a prerequisite for targeting, and saves about six weeks in the overall process.

According to this scheme, the targeting events can be recognized in cis. During P-induced gap repair and gene conversion, ectopic templates in cis are used more efficiently than templates on other chromosomes. The targeting efficiencies with donors in cis and in trans to the target locus are compared to determine the effects on efficiency.

It can be desirable to map the original transformant, and possibly keep it as a stock in case the targeting crosses were unsuccessful and needed repeating. But these steps can be carried out in tandem with the targeting screen. The main purpose of mapping is that, after targeting, the original (now unarked) insertion of TV2 can be crossed out. FLP and I-SceI elements can also be crossed out. The process can be simplified by choosing FLP and I-SceI insertions that are not on the target chromosome. Once a suitable targeting event is recovered, there is no longer a need to keep the original insertion.

Development of a Marker Segregation Vector

An alternative scheme involves generating a vector that carries two markers to visualize segregation of the original P element insertion and the targeting molecule. This vector has a structure similar to that of pTV2 (the plasmid clone of the TV2 vector) between the FRTs and can carry a second dominant marker outside the FRTs. The scheme to detect targeting relies on the dominant marker, which is included in the construct. Eye color markers are not well-suited to this scheme, but a reasonably good marker is the hybrid GMR-P35 gene [Hay et al.(1994) Development 120:2121-29]. This construct expresses the baculovirus P35 protein in the eye posterior to the morphogenetic furrow. The result is a moderate disorganization and roughening of the eye. After synthesis of FLP and I-SceI, targeting events are detected as progeny that are w⁺, but without rough eyes.

The present invention is not to be limited in scope by the specific embodiments described herein. The described embodiments are intended to be illustrative of individual ways that general aspects of the invention and functionally equivalent methods and components operate within the scope of the invention, including methods and components known in the art, whether or not they are specifically described or listed herein. Various modifications of the invention, in addition to those shown or described herein, will become apparent to those skilled in the art from the foregoing description and accompanying figures. Such modifications are intended to fall within the scope of the appended claims.

We claim:

1. A method of gene targeting in a transformable host organism comprising:
choosing a target gene of the host organism or portion thereof having known or cloned sequence,
transforming the host organism to contain an expressible gene encoding a unique endonuclease,
transforming the host organism to contain an excisable donor construct having a segment of sequence homologous to the target gene or portion thereof, the segment having a unique endonuclease site or sites inserted therein or adjacent to,
excising the donor construct and expressing the unique endonuclease, whereby a recombinogenic donor is produced, and
selecting for progeny of the host organism wherein recombination between the target and the recombinogenic donor has occurred.

2. The method of claim 1 wherein the endonuclease is expressed under control of an inducible promoter.

3. The method of claim 1 wherein the endonuclease is expressed under control of a tissue-specific promoter.

4. The method of claim 1 wherein the endonuclease is expressed under control of a ubiquitous, constitutive, or development stage-specific promoter.

5. The method of claim 3 wherein the promoter is a heat shock promoter.

6. The method of claim 3 wherein the promoter is inducible by the presence of a specified substance.

7. The method of claim 1 wherein the host organism is a multicellular organism or a single-celled organism.

8. The method of claim 7 wherein the host organism is an insect.

9. The method of claim 8 wherein the insect is a member of an insect order selected from the group Coleoptera, Diptera, Hemiptera, Homoptera, Hymenoptera, Lepidoptera, or Orthoptera.

10. The method of claim 9 wherein the insect is a member of the order Diptera.

11. The method of claim 10 wherein the insect is a fruit fly.

12. The method of claim 10 wherein the insect is a mosquito or a medfly.

13. The method of claim 1 wherein the host organism is a plant.

14. The method of claim 13 wherein the plant is a monocot.

15. The method of claim 14 wherein the plant is selected from the group consisting of maize, rice or wheat.

16. The method of claim 13 wherein the plant is a dicot.

17. The method of claim 16 wherein the plant is selected from the group consisting of potato, soybean, tomato, members of the Brassica family, or Arabidopsis.

18. The method of claim 13 wherein the plant is a tree.

19. The method of claim 1 wherein the host organism is a mammal.

20. The method of claim 19 wherein the mammal is selected from the group consisting of mouse, rat, pig, sheep, bovine, dog or cat.

21. The method of claim 1 wherein the host organism is a bird.

22. The method of claim 21 wherein the bird is selected from the group consisting of chicken, turkey, duck or goose.

23. The method of claim 1 wherein the host organism is a fish.

24. The method of claim 23 wherein the fish is a zebrafish, trout, or salmon.

25. The method of claim 1 wherein the donor construct is a target gene modifying sequence oriented with respect to the endonuclease site to provide ends-in recombination.

26. The method of claim 1 wherein the donor construct is a target gene modifying sequence oriented with respect to the endonuclease site or sites to provide ends-out recombination.

27. The method of claim 1 wherein the endonuclease is selected from the group consisting of rare-cutting endonucleases.

28. The method of claim 27 wherein the endonuclease is selected from the group consisting of I-SceI, I-TliI, I-Ceul, I-PpoI, I-CreI, or PI-PspI.

29. The method of claim 1 wherein the excisable donor construct comprises a pair of recombinase recognition sites flanking a segment of DNA comprising the segment of sequence homologous to the target gene, and the host cell contains a gene encoding a recombinase specific for said recombinase recognition sites.

30. The method of claim 29 wherein the recombinase is under expression control of an inducible promoter in the host cell, and the step of excising the donor construct comprises inducing the recombinase.

31. The method of claim 30 wherein the inducible promoter is a heat shock promoter.

32. The method of claim 30 wherein the inducible promoter is induced by the presence of a specified substance.

33. The method of claim 29 wherein the recombinase is under expression control of a tissue-specific promoter.

34. The method of claim 29 wherein the recombinase is under expression control of a development stage-specific promoter, a ubiquitous promoter, mRNA encoding recombinase, or recombinase protein.

35. The method of claim 29 wherein the recombinase and its specific recognition site, respectively, are selected from the group consisting of Cre and lox or Flp and FRT.

36. The method of claim 1 wherein the excisable donor construct comprises a pair of transposase recognition sites flanking a segment of DNA comprising the segment of sequence homologous to the target gene and the host cell contains a gene encoding the transposase specific for said transposase recognition sites.

37. The method of claim 1 wherein the excisable donor construct comprises DNA encoding one or more selectable markers.

38. The method of claim 37 wherein the selectable marker provides positive selection for cells expressing the marker.

39. The method of claim 37 wherein the selectable marker provides negative selection against cells expressing the marker.

40. The method of claim 37 wherein the selectable markers provide positive and negative selection of cells expressing the markers.

41. The method of claim 1 wherein the excisable donor construct comprises DNA encoding a screenable marker.

42. The method of claim 41 wherein the marker is selected from the group consisting of beta-glucuronidase, green fluorescent protein or luciferase.

43. The method of claim 1 wherein the step of transforming the host organism includes transforming a germ line cell of the host organism.

44. The method of claim 1 wherein the step of transforming the host organism consists essentially of transforming a somatic cell of the host organism.

45. A transformation vector comprising a target gene modifying sequence, the modifying sequence being homologous with a specified target gene or portion thereof, and having a unique endonuclease site inserted within the modifying sequence dividing said sequence into a first segment and a second segment.

46. The vector of claim 45 wherein the unique endonuclease site is selected from the group consisting of I-SceI, I-TliI, I-CeuI, I-PpoI or PI-PspI.

47. The vector of claim 45 wherein the first and second segments of the target gene modifying sequence are in parallel orientation with one another, whereby the vector is adapted for ends-in recombination.

48. The vector of claim 45 wherein the first and second segments of the target gene modifying sequence are in anti-parallel orientation with one another, whereby the vector is adapted for ends-out recombination.

49. The vector of claim 45 wherein the first and second segments of the target gene modifying sequence are in parallel orientation with one another, whereby the vector is adapted for ends-out recombination.

50. The vector of claim 45 additionally comprising a marker gene.

51. The vector of claim 50 wherein the marker gene encodes one or more selectable markers.

52. The vector of claim 50 wherein the selectable marker provides positive selection.

53. The vector of claim 50 wherein the selectable marker provides negative selection.

54. The vector of claim 50 wherein the selectable markers provide positive and negative selection.

55. The vector of claim 50 wherein the gene encodes a screenable trait.

56. The vector of claim 55 wherein the screenable trait is selected from the group consisting of beta-glucuronidase, green fluorescent protein or luciferase.

57. The vector of claim 45 further comprising a pair of recombinase recognition sites flanking a segment of DNA comprising the segment of sequence homologous to the target gene, and the host cell contains a gene encoding a recombinase specific for said recombinase recognition sites.

58. A method of gene targeting in a transformable host organism comprising:
choosing a target gene of the host organism or portion thereof having known or cloned sequence,
transforming the host organism to contain an expressible gene encoding a unique endonuclease,
transforming the host organism to contain a donor construct having a segment of sequence homologous to the target gene or portion thereof, the segment having a unique endonuclease site inserted therein,
expressing the unique endonuclease, whereby a recombinogenic donor is produced, and
selecting for progeny of the host organism wherein recombination between the target and the recombinogenic donor has occurred.

59. The method of claim 58 wherein the endonuclease is expressed under control of an inducible promoter.

60. The method of claim 58 wherein the endonuclease is expressed under control of a tissue-specific promoter.

61. The method of claim 58 wherein the endonuclease is expressed under control of a development stage-specific promoter.

62. The method of claim 60 wherein the promoter is a heat shock promoter.

63. The method of claim 60 wherein the promoter is inducible by the presence of a specified substance, an ubiquitous promoter, MRNA, or a protein.

64. The method of claim 58 wherein the host organism is a multicellular organism or a single-celled organism.

65. The method of claim 64 wherein the host organism is an insect.

66. The method of claim 64 wherein the insect is a member of an insect order selected from the group Coleoptera, Diptera, Hemiptera, Homoptera, Hymenoptera, Lepidoptera, or Orthoptera.

67. The method of claim 66 wherein the insect is a member of the order Diptera.

68. The method of claim 67 wherein the insect is a fruit fly.

69. The method of claim 67 wherein the insect is a mosquito or a medfly.

70. The method of claim 58 wherein the host organism is a plant.

71. The method of claim 70 wherein the plant is a monocot.

72. The method of claim 71 wherein the plant is selected from the group consisting of maize, rice or wheat.

73. The method of claim 70 wherein the plant is a dicot.

74. The method of claim 73 wherein the plant is selected from the group consisting of potato, soybean, tomato, members of the Brassica family, or Arabidopsis.

75. The method of claim 70 wherein the plant is a tree.

76. The method of claim 58 wherein the host organism is a mammal.

77. The method of claim 76 wherein the mammal is selected from the group consisting of mouse, rat, pig, sheep, bovine, dog or cat.

78. The method of claim 58 wherein the host organism is a bird.

79. The method of claim 78 wherein the bird is selected from the group consisting of chicken, turkey, duck or goose.

80. The method of claim 58 wherein the host organism is a fish.

81. The method of claim 80 wherein the fish is a zebrafish, trout, or salmon.

82. The method of claim 58 wherein the donor construct is a target gene modifying sequence oriented with respect to the endonuclease site to provide ends-in recombination.

83. The method of claim 58 wherein the donor construct is a target gene modifying sequence oriented with respect to the endonuclease site to provide ends-out recombination.

84. The method of claim 58 wherein the endonuclease is selected from the group consisting of rare-cutting endonucleases.

85. The method of claim 84 wherein the endonuclease is selected from the group consisting of I-SceI, I-TliI, I-CreI, I-CeuI, I-PpoI or PI-PspI.

86. The method of claim 58 wherein the donor construct comprises DNA encoding one or more selectable markers.

87. The method of claim 86 wherein the selectable marker provides positive selection for cells expressing the marker.

88. The method of claim 86 wherein the selectable marker provides negative selection against cells expressing the marker.

89. The method of claim 86 wherein the selectable marker provides positive and negative selection for cells expressing the marker.

90. The method of claim 58 wherein the donor construct comprises DNA encoding a screenable marker.

91. The method of claim 90 wherein the marker is selected from the group consisting of beta-glucuronidase, green fluorescent protein or luciferase.

92. The method of claim 58 wherein the step of transforming the host organism includes transforming a germ line cell of the host organism.

93. The method of claim 58 wherein the step of transforming the host organism consists essentially of transforming a somatic cell of the host organism.

94. The method of claim 58 wherein the step of transforming the host organism consists essentially of transforming a gamete cell of the host organism.
